(19) United States
(12) Patent Application Publication
(10) Pub. No.: US 2006/0190297 A1
(43) Pub. Date: Aug. 24, 2006

(54) Portable charge capture and inventory management

(76) Inventors: Larry Glass, South San Francisco, CA (US); Shailendrajeet Grewal, South San Francisco, CA (US); Stephen Hutchinson, South San Francisco, CA (US); Rajan Jena, South San Francisco, CA (US); Dan Lodder, South San Francisco, CA (US); Ann Tofolo, South San Francisco, CA (US); Berny Vandermolen, South San Francisco, CA (US)

Correspondence Address: COURTNEY STANIFORD & GREGORY LLP, P.O. BOX 9686, SAN JOSE, CA, 95157, US

(21) Appl. No.: 11/335,100

(22) Filed: Jan. 18, 2006

Related U.S. Application Data
(60) Provisional application No. 60/644,652, filed on Jan. 18, 2005.

(51) Int. Cl. G06Q 10/00

(52) U.S. Cl. 705/2

(57) ABSTRACT

Portable charge capture/inventory management systems and methods include a system comprising at least one server at a first location. The server of an embodiment includes inventory data and patient data. The inventory data includes information of drugs and supplies of a facility that is a patient treatment facility. The patient data includes identification, schedule and treatment data of patients of the facility. The system includes at least one portable client at the facility that is coupled to the server. The portable client is configured to access and present the patient data for use during treatment of each patient, and to capture transaction data of the treatment at a point of care. The transaction data is automatically transferred to the server.

### RELATED APPLICATION

This application claims the benefit of U.S. Patent Application No. 60/644,652, filed Jan. 18, 2005.

### TECHNICAL FIELD

The present invention relates generally to the field of medical inventory management and, more particularly, to portable charge capture and inventory management.

### BACKGROUND

Costs associated with patient health care have continued to increase. These costs include the costs of professional/administrative services as well as the costs of drugs and supplies used in patient treatment. As a result, office-based medical practices and treatment facilities face an increasing challenge in managing rising costs while providing consistently high-quality health care to their patients. Consequently there is a need for an efficient method of managing drug and supply inventories while capturing charges for services and drugs/supplies at the point of care.

### INCORPORATION BY REFERENCE

Each publication, patent and/or patent application mentioned in this specification is herein incorporated by reference in its entirety to the same extent as if each individual publication, patent and/or patent application was specifically and individually indicated to be incorporated by reference.

### BRIEF DESCRIPTION OF THE FIGURES

FIG. 1 is a block diagram of a portable charge capture/inventory management system, under an embodiment.

FIG. 1A is a flow diagram for portable charge capture/inventory management, under an embodiment.

FIG. 2 is a block diagram of a portable charge capture/inventory management system integrated with a practice having one or more inventory management machines, under an alternative embodiment.

FIG. 3 is a block diagram of a portable charge capture/inventory management system, under another alternative embodiment.

FIG. 4 is a patient treatment process that uses the Lynx Mobile system, under an embodiment.

FIG. 5 is a patient treatment process that uses the Lynx Mobile Web site, under an embodiment.

FIG. 6 is a dispense process that uses the Lynx Mobile system, under an embodiment.

FIG. 6A is a flow diagram for updating a dispense history using the Lynx Mobile system when dispensed items are returned to facility inventory, under an embodiment.

FIGS. 6B-6G show examples of select screens of the dispense process, under an embodiment.

FIGS. 6H-6K show examples of select screens of the dispense history updating, under an embodiment.

FIG. 7 is a flow diagram for generating a billing report (Super Bill) using the Lynx Mobile Web site, under an embodiment.

FIG. 8 is an example Super Bill Report Summary, under an embodiment.

FIG. 9 is an example Super Bill Report Detail, under an embodiment.

FIG. 10 is an example of a site login, under an embodiment.

FIG. 11 is an example operations screen of a Mobile Lynx Web site, under an embodiment.

FIG. 12 is another example operations screen of a Mobile Lynx Web site, under an embodiment.

FIG. 13 is yet another example operations screen of a Mobile Lynx Web site, under an embodiment.

FIG. 14 is an example System Administration screen of an Order Channel Web site, under an embodiment.

FIG. 15 is an example Facility Maintenance screen of an Order Channel Web site, under an embodiment.

FIG. 16 is a computer system for use in one or more of the Lynx Mobile system components, under an embodiment.

### DETAILED DESCRIPTION

A portable charge capture/inventory management system and method are described below. The portable charge capture/inventory management system and method, which is also referred to herein as the “Lynx Mobile system”, includes portable devices and auxiliary equipment along with the corresponding methods for charge capture and inventory management in an office-based or other medical practice (also referred to as a treatment facility or patient care facility). In particular, the Lynx Mobile system allows for the option of charge capture/inventory management in medical practices independent of inventory management machines or other inventory management equipment. While the description below describes the Lynx Mobile system in a medical practice, alternative embodiments can be used in other environments in which charge capture/inventory management systems and methods are needed.

The following description provides specific details for a thorough understanding of, and enabling description for, embodiments of a portable charge capture/inventory management system and method. However, one skilled in the art will understand that the portable charge capture/inventory management systems and methods described herein may be practiced without these details. In other instances, well-known structures and functions have not been shown or described in detail to avoid unnecessarily obscuring the description of the embodiments of the portable charge capture/inventory management system and method.

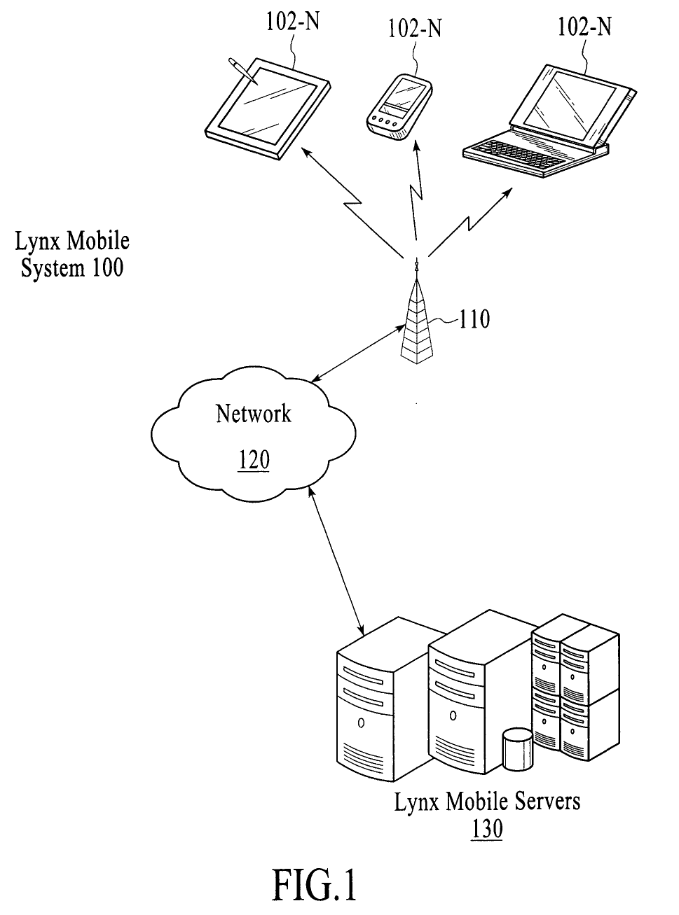

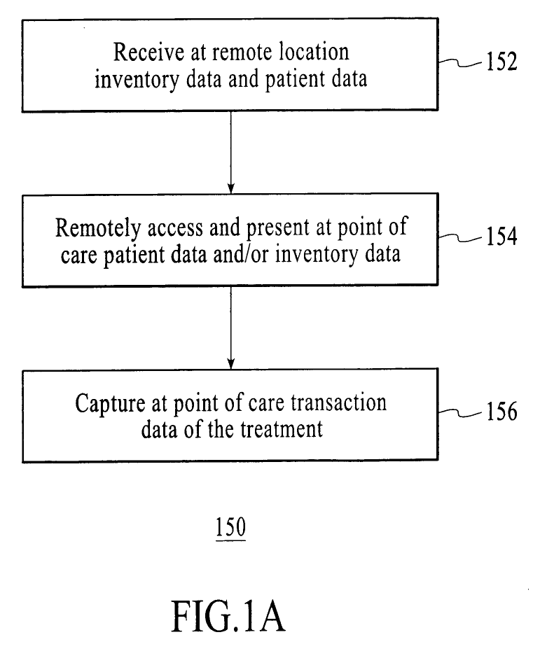

FIG. 1 is a block diagram of a portable charge capture/inventory management system 100, under an embodiment. This embodiment of the Lynx Mobile system 100 includes one or more portable or mobile devices 102-N (where N represents any one of a number of portable devices 0, 1, 2 . . . X) that couple to at least one access point 110 or wireless access point 110. The access point 110 couples to one or more Lynx Mobile servers 130 via couplings with one or more networks 120. The servers 130 may host an Internet site (also referred to herein as the Lynx Mobile Web site, or the Web site) by which a user of one of the portable devices 102-N and/or the access point 110 may access information of the servers 130.

Alternative embodiments of the Lynx Mobile system 100 may include any number of portable devices 102-N, access points 110, networks 120, and/or servers 130. Further, the Lynx Mobile system 100 of various alternative embodiments may not include one or more of the access points 110 and networks 120 and/or may distribute the functionality of the access points 110 and/or the networks 120 described herein among other components of the system (e.g., portable devices 102-N, servers 130, etc). Moreover, the portable devices 102-N of various alternative embodiments may couple to the servers 130 without a coupling to the access points 110 and/or the network 120.

The servers 130 of an embodiment generally include and provide access to a variety of information including, but not limited to, patient data or information, inventory information, schedule information, and practice information. As such, the Lynx Mobile portable devices 102-N operate together with the Lynx Mobile servers 130 to integrate inventory management and charge capture with the patient care process by providing portable charge capture and inventory management at the point of care of patients, as described below. The charge capture includes capture of charges for drugs, supplies, professional services, administrative services, as well as other types of supplies, equipment and services appropriate to a medical office or practice.

The portable devices 102-N, access points 110, and/or servers 130 include but are not limited to processor-based devices like, for example, portable computers (PCs), portable computing devices, personal digital assistants (PDAs), Tablet PCs, communication devices, cellular telephones, portable telephones, portable communication devices, and subscriber devices or units. The portable devices 102-N, access points 110, and/or servers 130 can include all such devices and equivalents, and are not limited to any particular type of processor-based electronic device.

As described above, each of the portable devices 102-N of an embodiment communicates with the servers 130 using at least one coupling over at least one network 120. The network 120 includes various network components (not shown) of one or more communication service providers or carriers, but is not so limited. Further, the network 120 and corresponding network components can be any of a number/combination of network types known in the art including, but not limited to, the Internet, proprietary networks, local area networks (LANs), metropolitan area networks (MANs), wide area networks (WANs), wireless networks and backend networks for example. Additionally, the network 120 may include a hybrid network that uses the Internet for some portion of the communications routing, for example, while using one or more different networks for other portions of the communications routing. The couplings between each of the portable devices 102-N and the servers 130 may include one of wired, wireless, and hybrid wired/wireless couplings or connections, and may use any number of communication systems and protocols known in the art.

A more specific example of the Lynx Mobile system 100 includes a portable handheld device 102-N for use by medical professionals and other personnel at a patient treatment facility. The portable device 102-N couples to at least one server 130, where the server is at a location different or remote to that of the portable device 102-N. Components of the server include or store inventory data and/or patient data of the treatment facility. The inventory data includes information of drugs and supplies of the treatment facility, for example, including but not limited to inventory levels of drugs and supplies available at the facility along with descriptions, product codes, and other relevant information of the drugs and supplies. The patient data includes patient identification data, patient schedules and treatment data or regimens of patients of the host treatment facility as well as billing data relating to any treatments the patient receives at the facility. The inventory data and/or the patient data may include other data as appropriate to operations of the host facility. An example of this system is the Lynx Mobile system available from Oncology Therapeutics Network of South San Francisco, Calif.

The portable device 102-N is a handheld device that is configured to allow users to access, view and/or record patient data for use during treatment of each patient. The patient data is permanently stored on the remote server and/or some other component of the Lynx Mobile system or the host facility, but the embodiment is not so limited. The portability of the portable device allows personnel at a treatment facility to use the device whether they are for example at a nurse's station or in an infusion room. The portable device 102-N may also allow users to access and view inventory data of the treatment facility.

The portable device 102-N is further configured to capture transaction data of treatment procedures administered to a patient at a point of care. The capture includes data recorded or entered into the portable device 102-N by a user as well as data entered via other methods (e.g., scanned using a scanner coupled to the portable device 102-N). The transaction data includes but is not limited to data of one or more of drugs and supplies dispensed during the treatment, data of one or more of unused drugs and unused supplies resulting from the treatment, drugs and supplies wasted during the treatment, professional services of the treatment, administrative services of the treatment, current procedural terminology (CPT) codes of the treatment, evaluation and management (E&M) codes of the treatment, product codes of one or more of the drugs and supplies dispensed during the treatment. Transaction data may include any other type of data as appropriate to operations of the host facility.

The portable device 102-N automatically transfers the transaction data to the server and/or other component of the Lynx Mobile system or facility simultaneous with or subsequent to capture. The server is configured to automatically update the inventory data and/or the patient data using the transaction data received from the at least one portable client. Therefore, the portable device 102-N allows facility personnel like nurses and/or physicians to access patient data at point and time of care for the patient being treated and, in addition, to record or capture transaction data of the treatment at point and time of care.

The portable device of alternative embodiments may support access to data storage devices of the facility other than the Lynx Mobile servers 130. Also, the portable device of alternative embodiments may operate as a distributed storage device of the servers 130 so that components of the portable device include or store inventory data and/or patient data of the treatment facility. Furthermore, the portable device of alternative embodiments may include patient data or inventory data by temporarily caching or storing the data.

In addition to being configured to automatically update the inventory data and/or the patient data using the transaction data received from the at least one portable client, the server is configured to support other operations of the treatment facility. For example, the server of an embodiment is configured to automatically order drugs and/or supplies for the facility in response to the inventory data and/or the patient data. When the patient data described above includes billing data of treatments received by a patient, the server is configured to generate the billing data using the transaction data. The server is further configured to automatically generate bills and/or any number of types of billing reports for the facility in response to the inventory data and/or the patient data.

The Lynx Mobile system also includes a secure portal by which a facility accesses and manipulates inventory data and patient data of the facility. The Lynx Mobile system portal of an embodiment includes a Lynx Mobile Web site hosted at the server, but is not limited to this Web site. Personnel at the host treatment facility can access inventory data and patient data stored on the server in order to view and manipulate (e.g., add, delete, revise, etc.) the data as described in detail below. The Web site, for example, allows facility personnel to access facility data stored on the server according to any number of categories including, for example, dispensing, queuing, inventory, patients, reports, and administration to name a few. The Web site is configured to allow manipulation of the patient data by allowing facility personnel to generate and/or select dispense profiles for use in dispensing drugs and/or supplies during patient treatment.

FIG. 1A is a flow diagram 150 for portable charge capture/inventory management, under an embodiment. Using the Lynx Mobile system 100 as an example, operations 150 include receiving 152 at a first location the inventory data and patient data as described above. The data is received via any number of devices or methods and can be stored at the first location or at another location remote to the first location. The first location of an embodiment includes the Lynx Mobile server(s) described above at a remote location but is not so limited. The patient data and/or the inventory data are accessed and presented 154 at a point of care in the facility for use during treatment of each patient. Access to and presentation of the data at the point of care is via at least one portable handheld device and a coupling to the first location. Transaction data of the treatment are captured 156 at the point of care during the treatment. The capturing includes facility personnel entering and/or scanning the transaction information via the portable device. The captured transaction data is automatically transferred to the first location as described above.

The Lynx Mobile system described herein thus provides an easy-to use handheld device that can be carried by personnel to capture charges at the point of care. The system supports standardized regimens with services and treatment-related charges. Because the Lynx Mobile system is fully integrated into a provider's ordering and reporting processes, if provides efficient management of inventory levels along with automation of the drug and supply ordering process. Further, patient treatment histories and charges are updated in real time, so reporting capabilities are always up to date.

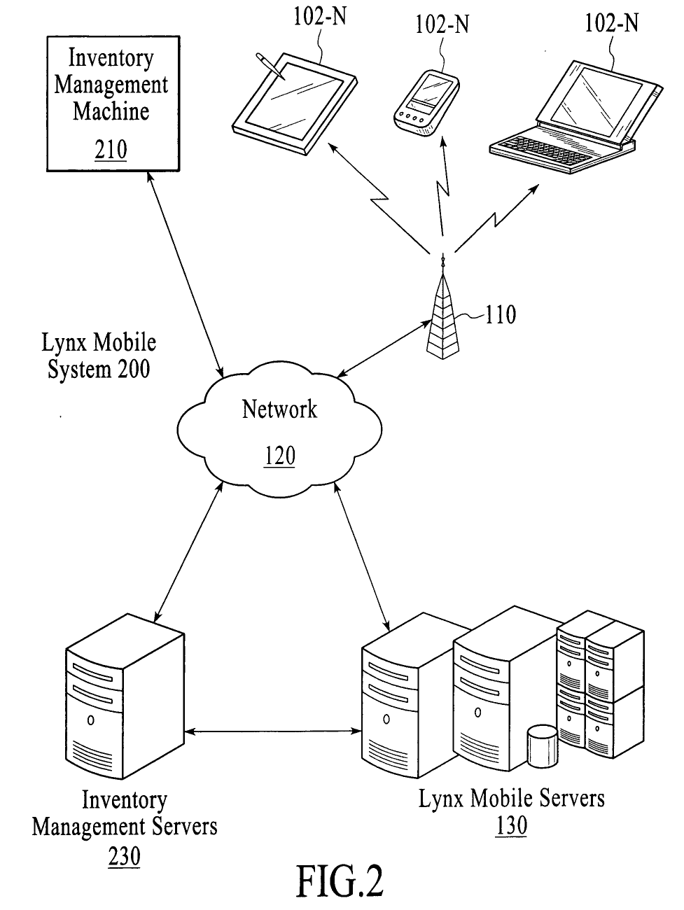

FIG. 2 is a block diagram of a portable charge capture/inventory management system 200 integrated with a practice having one or more inventory management machines 210, under an alternative embodiment. This Lynx Mobile system 200 includes one or more portable or mobile devices 102-N that couple to at least one access point 110 or wireless access point 110. The access point 110 couples to one or more Lynx Mobile servers 130 via couplings with one or more networks 120. The system 200 also includes one or more inventory management machines 210 (also referred to as inventory devices 210) that couple to communicate with one or more portable or mobile devices 102-N via couplings with the networks 120. The Lynx Mobile servers 130 and the inventory management machines 210 couple to communicate using one or more couplings that include networks 120, for example, or other backend or proprietary networks (not shown). The servers 130 may host an Internet site by which a user of one of the portable devices 102-N and/or the inventory management machine 210 may access information of the servers 130. Alternative embodiments may include any number of portable devices 102-N, inventory management machines 210, access points (not shown), networks 120, and/or servers 130.

The inventory device(s) 210 at the facility is coupled to the portable device 102-N and/or the server 130. The inventory device 210 of an embodiment is configured to contain and/or dispense drugs and supplies for use in patient treatments. The inventory device 210, when present, is integrated into the Lynx Mobile system and operates automatically in response to information received from the portable device 102-N via a direct or indirect coupling with one or more other components of the Lynx Mobile system (e.g., the server 130, inventory management server 230, etc.) and/or other components of the host treatment facility. As an example, a user controls locking/unlocking of the inventory device 210 via information entered at the portable device 102-N and a coupling (e.g., wireless, infrared, Bluetooth, etc.) with the inventory device 210. Alternatively, functions of the inventory device 210, when present, are controlled in response to information received from the server 130 and/or the inventory management server 230.

Alternative embodiments of the Lynx Mobile system 200 may include any number of portable devices 102-N, access points 110, networks 120, inventory management machines 210, and/or servers 130. Further, the Lynx Mobile system 200 of various alternative embodiments may not include one or more of the access points 110, networks 120, and inventory management machines 210 and/or may distribute the functionality of the access points 110, networks 120, and/or inventory management machines 210 described herein among other components of the system (e.g., portable devices 102-N, servers 130, etc). Moreover, the portable devices 102-N of various alternative embodiments may couple to the servers 130 without a coupling to the access points 110 and/or the network 120.

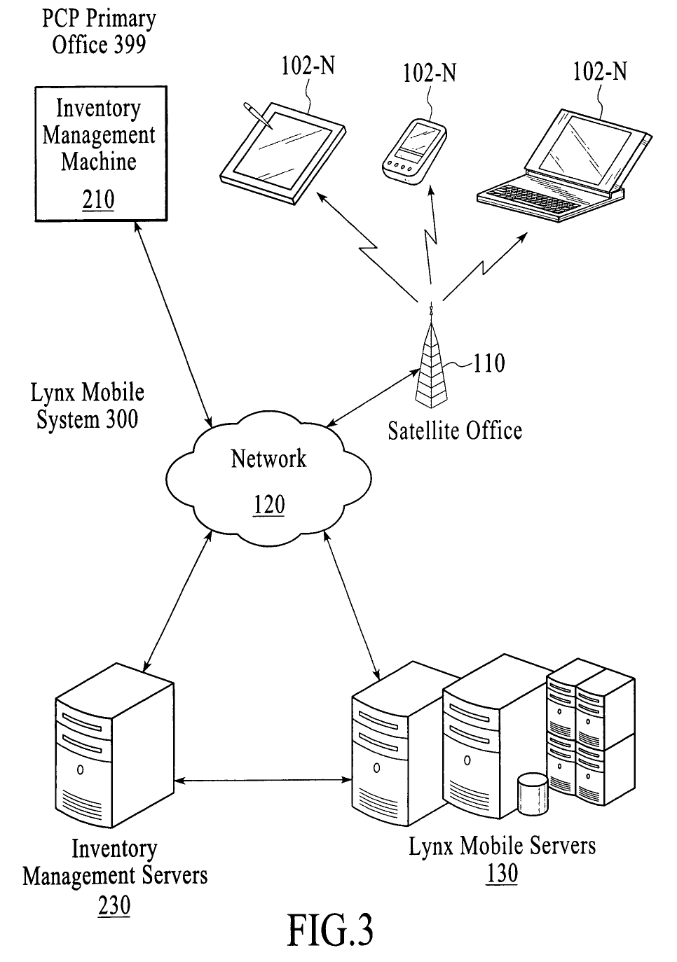

FIG. 3 is a block diagram of a portable charge capture/inventory management system 300 integrated with a practice having a primary facility 399 and one or more satellite facilities, under another alternative embodiment. This Lynx Mobile system 300 includes one or more inventory management machines 210 located at the care provider's primary facility 399. As described above, the inventory management machines 210 communicate with one or more inventory management machine servers 230 via couplings with the network 120. The Lynx Mobile system 300 also includes at least one portable or mobile device 102-N that couples to at least one access point 110, where the Lynx Mobile portable device 102-N and the access point 110 are located at one or more satellite offices of the care provider's primary facility 300. The portable devices 102-N may be in use at any of a number of the primary 399 and/or satellite offices. The access point 110 couples to one or more Lynx Mobile servers 130 via couplings with one or more networks 120. The inventory management machine servers 230 couple to one or more Lynx Mobile servers 130 via direct couplings and/or couplings with one or more networks 120. Alternative embodiments may include any number of satellite offices, portable devices 102-N, inventory management machines 210, access points (not shown), networks 120, and/or servers 130.

Alternative embodiments of the Lynx Mobile system 300 may include any number of portable devices 102-N, access points 110, networks 120, inventory management machines 210, and/or servers 130. Further, the Lynx Mobile system 300 of various alternative embodiments may not include one or more of the access points 110, networks 120, and inventory management machines 210 and/or may distribute the functionality of the access points 110, networks 120, and/or inventory management machines 210 described herein among other components of the system (e.g., portable devices 102-N, servers 130, etc). Moreover, the portable devices 102-N of various alternative embodiments may couple to the servers 130 without a coupling to the access points 110 and/or the network 120.

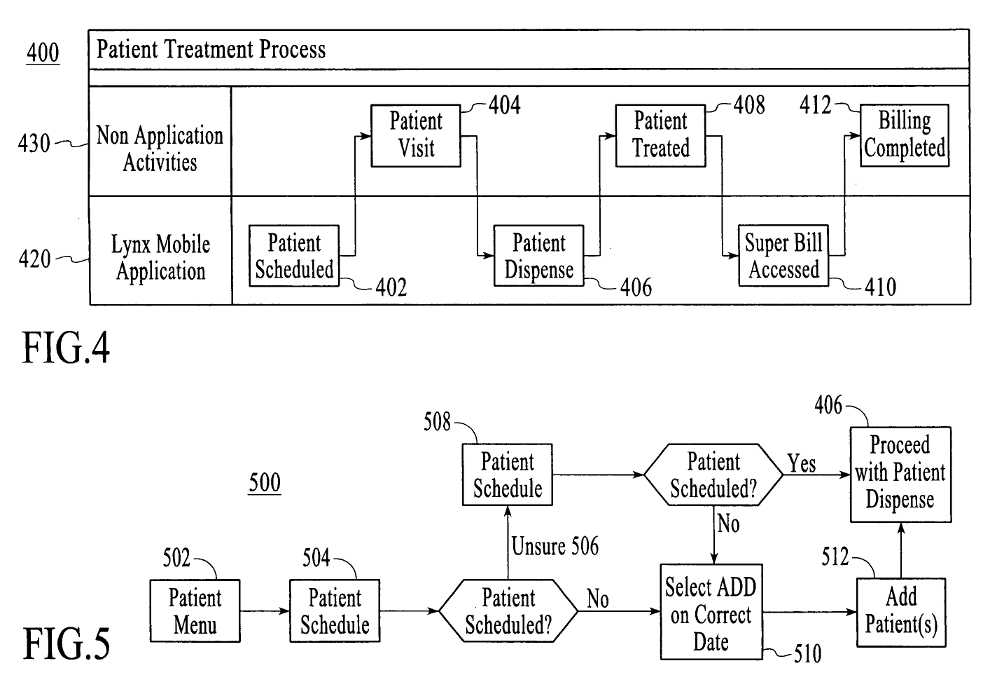

FIG. 4 is a patient treatment process 400 that includes the Lynx Mobile system, under an embodiment. Each of the Lynx Mobile systems 100, 200, 300 described above operates under the patient treatment process 400, but is not so limited. The patient treatment process is described below according to those activities 420 involving use of a component of the Lynx Mobile system and those activities 430 not involving use of a Lynx Mobile system component. The patient treatment process 400 begins when the patient is scheduled 402 for a treatment date on, for example, the Lynx Mobile Web site. The scheduling is done on the Lynx Mobile Web site of an embodiment using the Patient Menu under Patient Schedule, as described below. The patient visits 404 the practice facility for treatment on the scheduled date. Drugs and supplies are dispensed 406 before and/or simultaneous with the patient visit using components of the Lynx Mobile system, and the dispensing is further described below. The patient is treated 408 at the facility using the dispensed items, where the dispensed items include drugs, supplies, professional services of the treatment, and administrative services of the treatment.

The patient treatment process 400 also includes billing activities for the treatment. The Lynx Mobile system of an embodiment supports regular periodic access to and manipulation of billing records of patients of the facility by an employee at the treatment facility. For example, the Lynx Mobile system provides access 410 to a billing report (referred to herein as a Super Bill) at the end of the day that includes billing information of patients treated that same day at the facility; alternative embodiments can use any period of time for access and are not limited to access once a day or at the end of a day. Access 410 to the billing report is via the Lynx Mobile Web site under the report menu in an embodiment, but is not so limited. The accounting or billing department of the treatment facility uses the billing reports to bill 412 for reimbursement of costs associated with each treatment activity and/or for submitting claims for reimbursement to other organizations (e.g., insurance).

The patient scheduling 402 of the patient treatment process of an embodiment uses the Lynx Mobile Web site; alternative embodiments may schedule patients through any of a number of other components of the Lynx Mobile system and/or interfaces in the art. FIG. 5 is a patient treatment process 500 using the Web site, under an embodiment. The Web site includes for example a Patient Menu 502 that includes or provides access to three areas: a Patient List; a Patient Search; and a Patient Schedule 504. The Patient List includes at least one area or Web page for managing patients and their information. The Patient Search allows the user to search for patients within the system.

The Patient Schedule 504 is an area of the Web site where a user schedules the patients for their treatment date. Patients scheduled for treatment on a particular day will show up on the Lynx Mobile system under the dispense menu for treatment that day. If a user of the Lynx Mobile system is unsure 506 whether a patient is scheduled for treatment on a particular day, the user navigates (e.g., selects a Patient Link on the Web page) to patient information 508 that includes information of when a particular patient is scheduled for treatment. When the patient is scheduled for treatment the user proceeds with dispensing 406 for the treatment, as described above.

When a patient is not scheduled for treatment, the user of the Lynx Mobile system adds the patient to the schedule (e.g., selects 510 an “ADD” icon on the displayed Web page in order to add the patient to the schedule). Taking action to add the patient (e.g., selecting “ADD” on the appropriate date) results in display of a list of the patients that are in the Lynx Mobile database for the treatment facility. The user places a check mark next to the patient names or otherwise selects the patients that are to be added to the schedule for that day and takes action as appropriate to the user interface to add the selected patients to the schedule for that day (e.g., selects an icon or button of the Web page to add 512 the checked or selected patients to the treatment schedule for that day). Once the patients have been added to the treatment schedule for the day the user proceeds with dispensing 406 for the treatment, as described above.

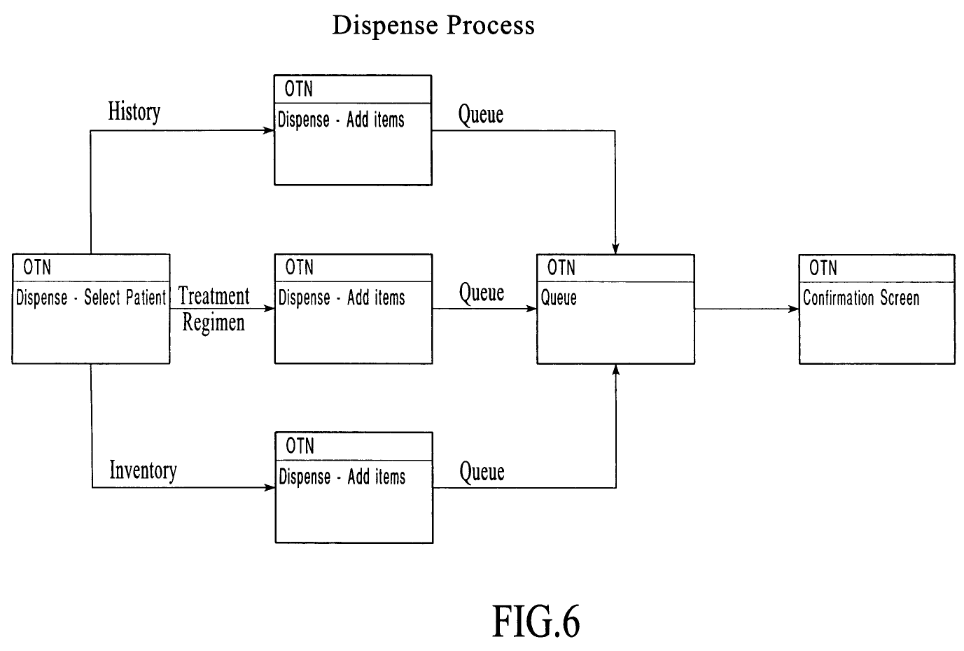

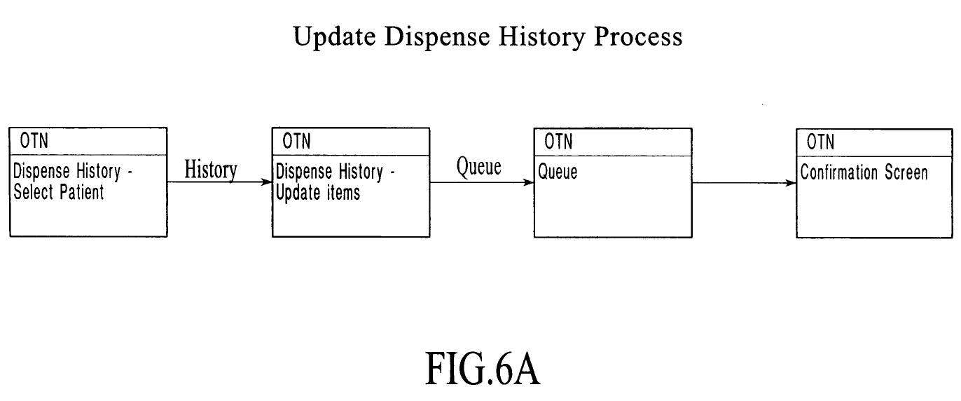

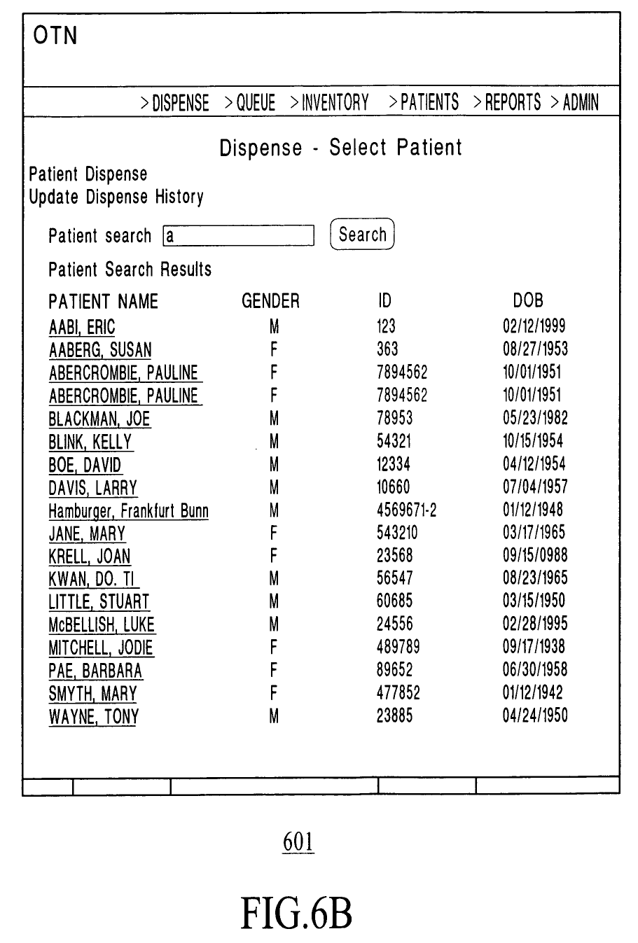

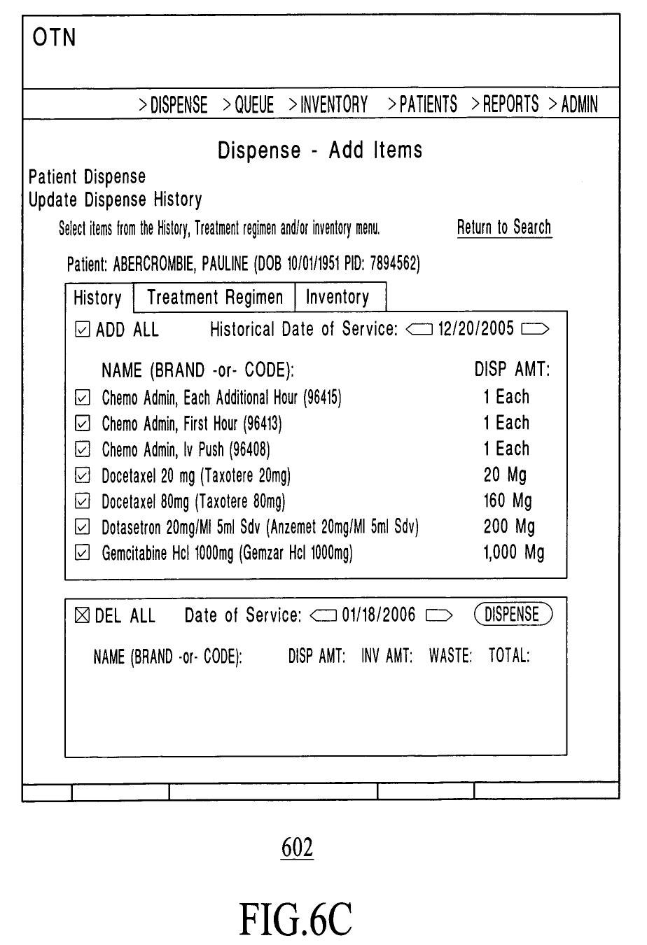

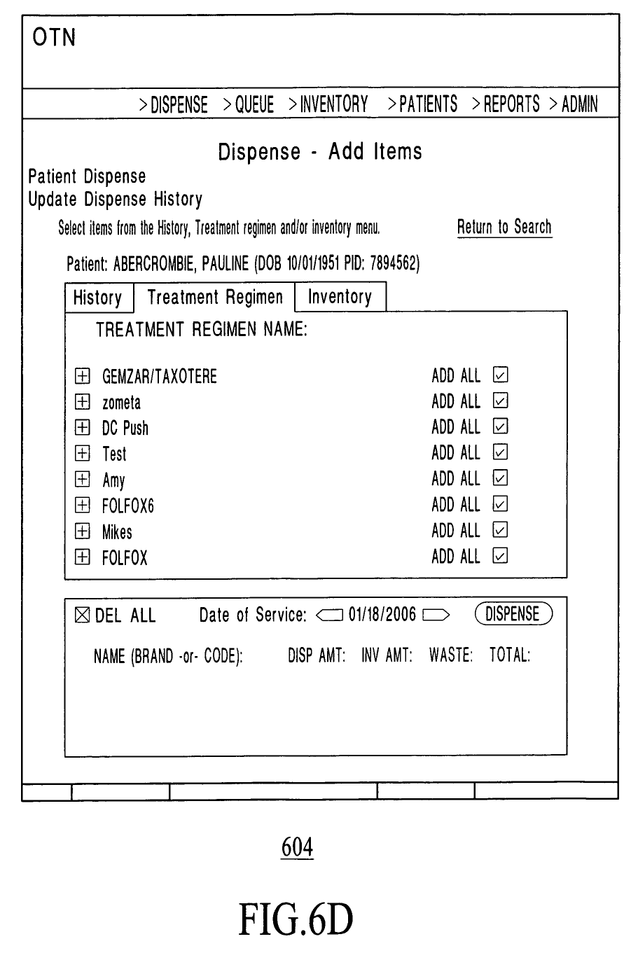

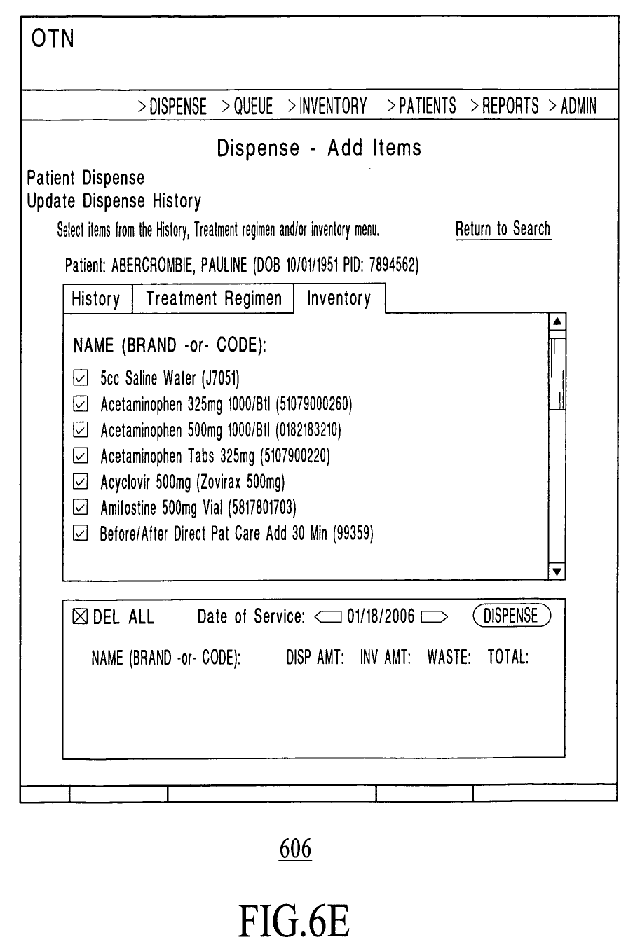

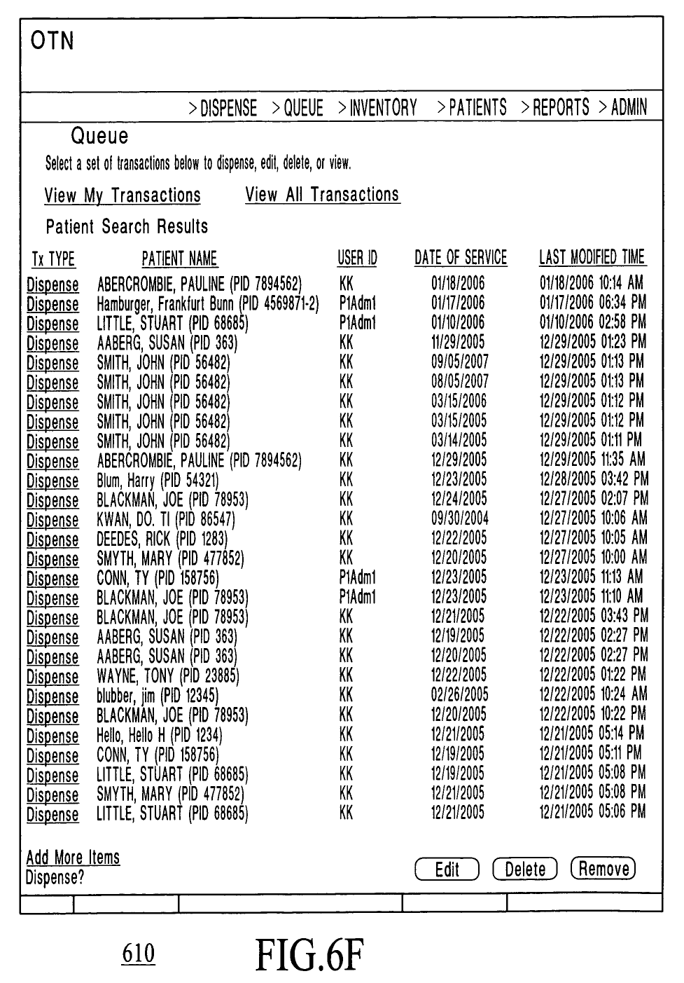

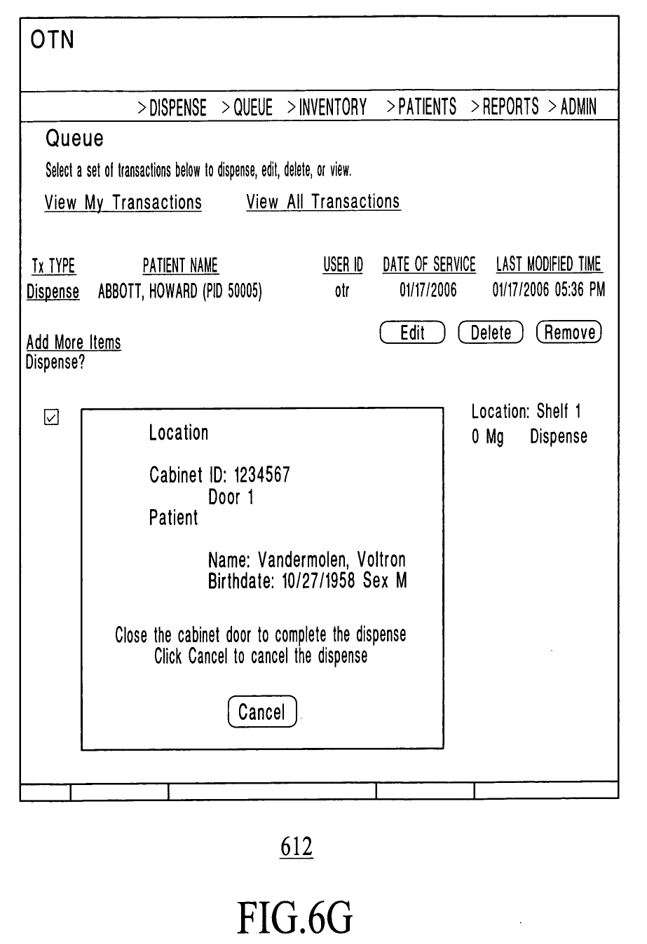

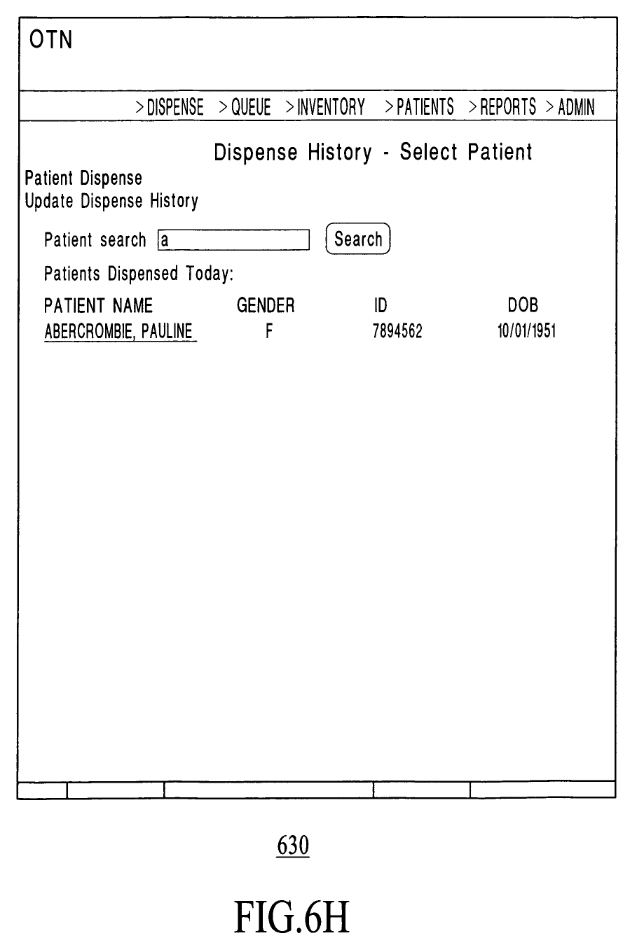

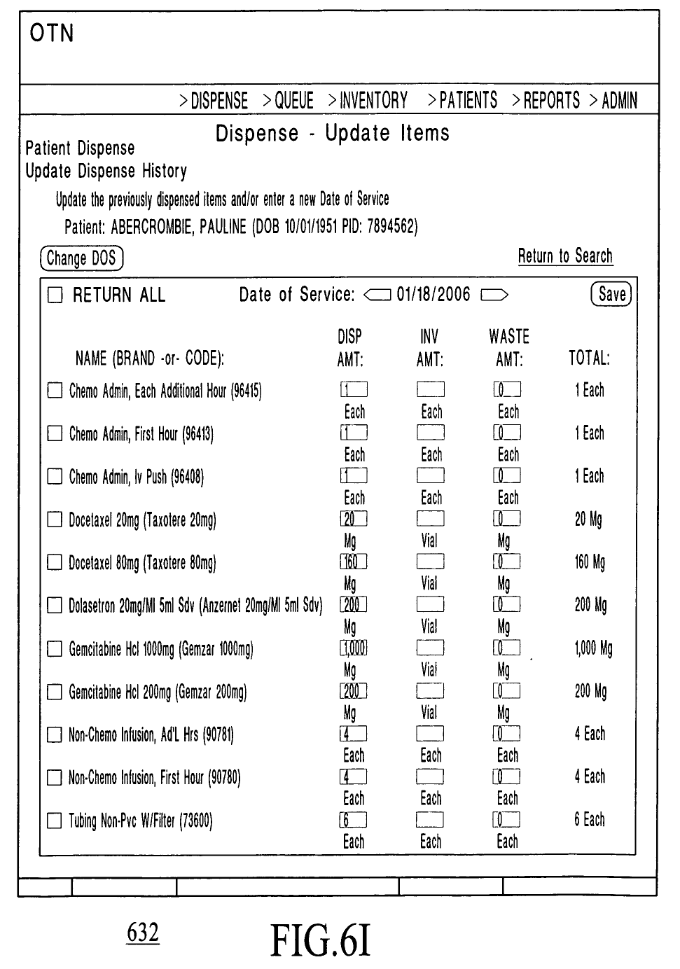

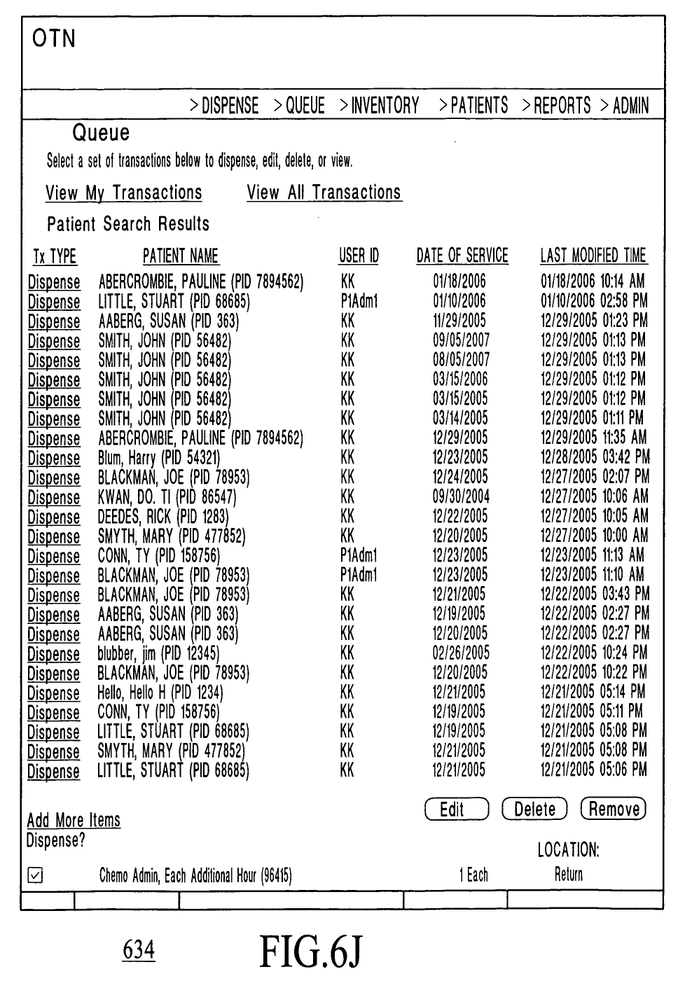

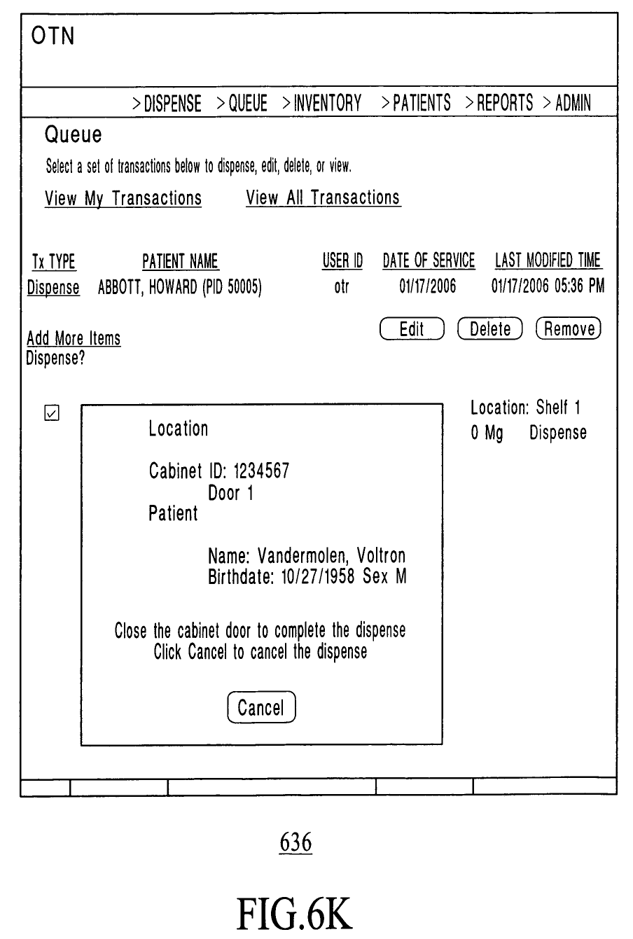

FIG. 6 is a dispense process 600 using the Lynx Mobile system, under an embodiment. FIGS. 6B-6G show examples of select screens of the dispense process 600, under an embodiment. The dispense process 600 begins with selection of a patient 601. Following patient selection 601, the dispense process 600 of an embodiment allows a user to select items for dispensing according to patient history 602, patient treatment regimens 604, or an inventory item list 606, each of which are described in detail below. The dispense process 600 of the patient treatment process of an embodiment uses the Lynx Mobile Web site; alternative embodiments may dispense items through any of a number of other components of the Lynx Mobile system and/or interfaces in the art.

A user can select items to be dispensed during treatment using information of a patient's treatment history 602. The selection of items according to treatment history proceeds with the selection of a patient to be treated (e.g. Select Patient via on Web page). The date of service is then selected as the date on which the selected items will be dispensed (e.g. Select DOS via Web page). The user then selects items in the patient's treatment history that are to be dispensed (e.g. according to History Item Name on Web page). The user enters the quantity for each item to be dispensed (if needed) (e.g. Edit Item via Web page), and selects dispense. The user navigates to a Queue screen 610 and confirms 612 the selections to complete the transaction (e.g. Confirm icon on Web page).

A user can also select items to be dispensed during treatment using information of a patient's treatment regimens 604. The selection of items according to treatment regimen proceeds with the selection of a patient to be treated (e.g. Select Patient on Web page). The treatment regimen list is then selected as the treatment regimen to be dispensed to the patient (e.g. Treatment Regimen icon on Web page). The user then selects items in the patient's treatment regimen that are to be dispensed (e.g. Treatment Regimen Item Name via Web page). The user enters the quantity for each item to be dispensed (e.g. Edit Item via Web page), and selects dispense. The user navigates to a Queue screen 610 and confirms 612 the selections to complete the transaction (e.g. Confirm icon on Web page).

A user can further select items to be dispensed during treatment using information of an inventory item list 606. The selection of items according to an item list proceeds with the selection of a patient to be treated (e.g. Select Patient on Web page). The user then selects or scans items that are to be dispensed (e.g. Item List via Web page). The user enters the quantity for each item to be dispensed (e.g. Select Item on Web page), and selects dispense. The user navigates to a Queue screen 610 and confirms 612 the selections to complete the transaction (e.g. Confirm icon on Web page).

Following dispense operations, a user updates a patient dispense history. FIG. 6A is a flow diagram for updating 620 a dispense history using the Lynx Mobile system when dispensed items are returned to facility inventory, under an embodiment. FIGS. 6H-6K show examples of select screens of the dispense history updating 620, under an embodiment. The dispense history updating of an embodiment uses the Lynx Mobile Web site; alternative embodiments may update the dispense history through any of a number of other components of the Lynx Mobile system and/or interfaces in the art.

The updating 620 begins with the selection 630 of a patient for whom items will be returned (e.g. Select Patient on Web page). The date of service is then selected for the items to be returned (e.g. Select DOS via Web page). The user then selects 632 items in the patient's treatment history that are to be returned (e.g. History Item Name on Web page). The user enters the quantity for each item to be returned (if needed) (Edit Item on Web page), and takes an action to save the information as appropriate to the user interface (e.g. selects “SAVE” on Web page). The user navigates to a Queue screen 634 and confirms 636 the selections to complete the transaction (e.g. Confirm icon on Web page).

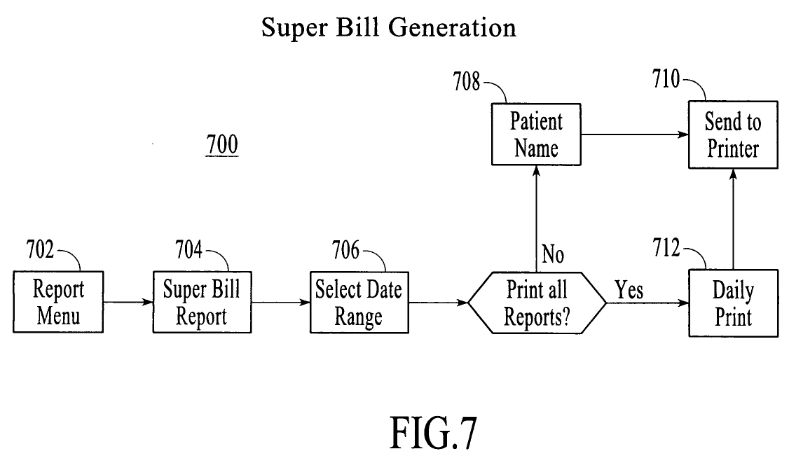

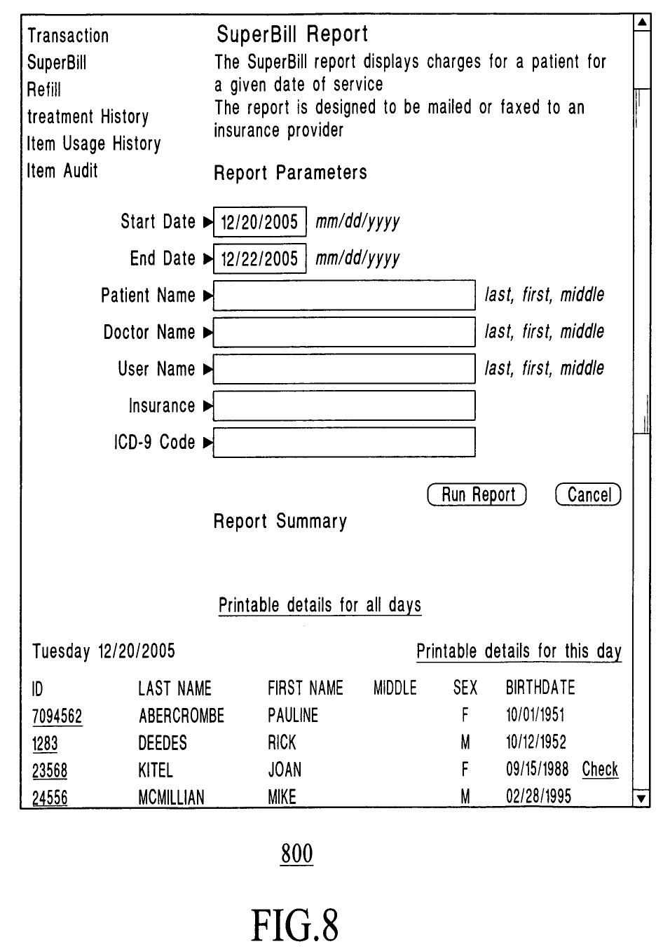

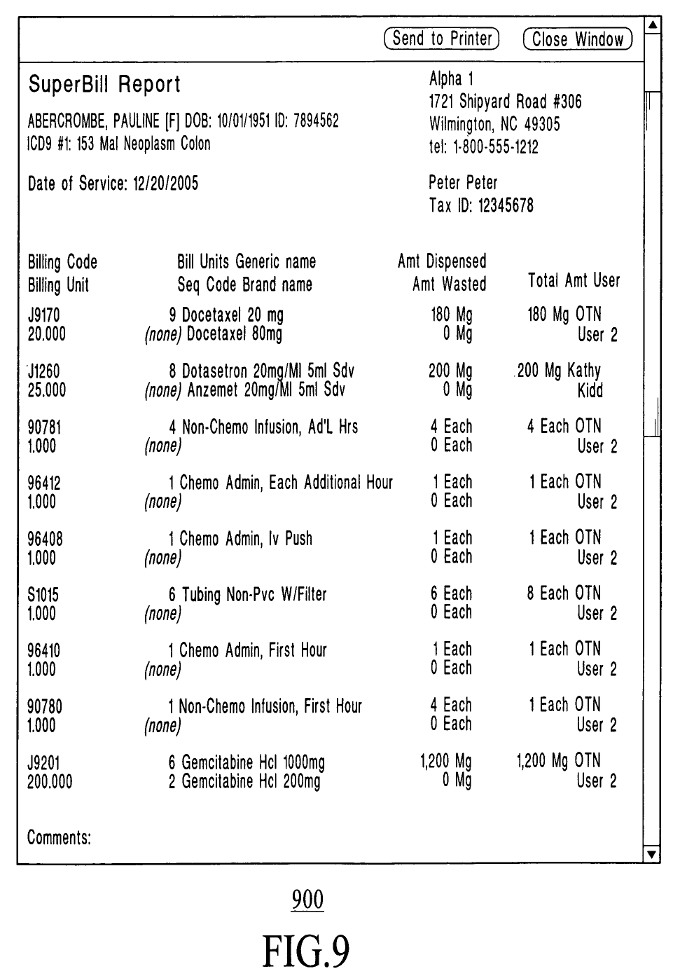

The patient treatment process 400 as described above includes billing activities for patient treatments. The Lynx Mobile system of an embodiment supports regular periodic access to a billing report referred to as a Super Bill that includes billing information associated with patient treatments at the facility. FIG. 7 is a flow diagram for generating 700 a billing report (Super Bill) using the Lynx Mobile Web site, under an embodiment. The Super Bill generation of an embodiment uses the Lynx Mobile Web site; alternative embodiments may generate billing information and/or reports through any of a number of other components of the Lynx Mobile system and/or interfaces in the art. The Super Bill Report is a replacement for the super bill that is utilized by the practice for reimbursement. The Super Bill Report displays the charges, drugs and supplies that will be charged to a patient. The Super Bill Report includes the billing code, billable unit, the item name, amount dispensed to the patient, amount wasted and the total amount. The bill units are calculated by dividing the total amount by the billable units. Sample reports are shown elsewhere herein. FIG. 8 is an example Super Bill Report Summary 800, under an embodiment. FIG. 9 is an example Super Bill Report Detail 900, under an embodiment.

In generating a Super Bill Report, a user on the Lynx Mobile Web site navigates to a Report Menu 702. The Report Menu 702 includes but is not limited to a Super Bill Report, a Transaction Report, a Refill Report, a Treatment History Report, an Item Usage Report, and an Item Audit Report. Additional reports can be added to the Lynx Mobile Web site as appropriate to treatments and treatment facilities. The user desiring a Super Bill Report selects a Super Bill Report 704 link on the Report Menu 702. The user then enters or selects 706 a date range for the Super Bill Report. The Super Bill Report can be filtered according to any of a number of parameters, but is not so limited. As an example, the Super Bill Report can be filtered according to one or more of patient name, physician name, user name, insurance, and the International Classification of Diseases, Ninth Revision (ICD-9) to name a few. When the user elects to print a Super Bill Report for a specific patient, the user selects 708 the patients name link and sends 710 the information to the printer. When the user elects to print a Super Bill Report for all patients treated during a single day, the user selects 712 a daily print and sends 710 the information to the printer. The user can also elect to print the Super Bill Reports for treatments during a specified date range.

The Lynx Mobile system, as described above, includes servers along with portable handheld devices and a Web site that provide remote access to inventory and patient information of a treatment facility. The Lynx Mobile system is multi-tiered, with numerous functionally-independent layers arranged in a stack. These layers include but are not limited to one or more of the following: hardware, operating system, database, application server object-relational mapping, application server business logic, application server user interface, web server, and client web browser layers. Each layer attempts to completely abstract (e.g., from the layer built upon it) the layer on which it is built. While the embodiments described herein may include specific examples of hardware and/or software components, along with specific examples of couplings and/or connections between components, the Lynx Mobile system is not limited to operating with/under these components and is not limited to the embodiments described herein.

The Lynx Mobile system operating system abstracts the details of communicating with the hardware and provides a user interface for administration. The hardware for the Lynx Mobile server of an embodiment is available from Sun Microsystems for example, running Sun Solaris 8 OS, but is not limited to this hardware. The database layer provides advanced support for storage and retrieval of data. The Lynx Mobile database is running on an Oracle 9i instance for example, but is not so limited. The application server takes data from the database (object-relational mapping layer), characterizes actions on that data in terms of business processes (business logic layer), and provides a dynamic web interface for users to participate in these business processes (user interface layer). The Lynx Mobile application server is Weblogic Server 8.1.3, an implementation of the Java 2 Enterprise Edition, but is not limited to a Weblogic Server. The Lynx Mobile system also includes a simple web server proxy, iPlanet, used for HyperText Transfer Protocol Secure (HTTPS) secure web communication. A firewall structure and/or other security mechanisms in the art protect the Lynx Mobile infrastructure and the client data from malicious use.

The Lynx Mobile system exposes the Lynx Mobile Web site, which in an embodiment comprises an HTTPS web application user interface. The Web site can be used on any computer with an Internet Explorer 4.0+ equivalent browser. The Web interface includes an administration functionality used to configure users, permissions, doctors, site details, products, patients, and scheduling, and generate reports. The Web site also allows the user to Dispense and Return inventory items. The Web site uses the HTTPS protocol for secure communication between the server and the clients over the World Wide Web.

The Lynx Mobile system provides content in the form of visual information for use by one or more of system administrators, practice administrators, practice billing personnel, and health care providers to name a few. The Lynx Mobile system also provides content in the form of Extensible Markup Language (XML)-formatted Web Services information, such as Inventory Refill Requests, and Web services used to interface the Lynx Mobile system to any number/type of external systems.

The Lynx Mobile Web site of an embodiment includes the use of Java (J2SE, J2EE, and JSP) and Microsoft Windows 2000 Server (Operating System), but is not so limited and can use any type and/or combination of other technologies in the art. Note that implementation details are provided herein as examples only and are inconsequential to the overall design, and the Web page examples that follow are provided as examples only as the Lynx Mobile Web site is not limited to these presentations. For example, any major Java Application server can be used to host the application. Likewise, any major relational database management system (RDBMS) can be used. Similarly, the design is not dependent on Microsoft Windows 2000 Server.

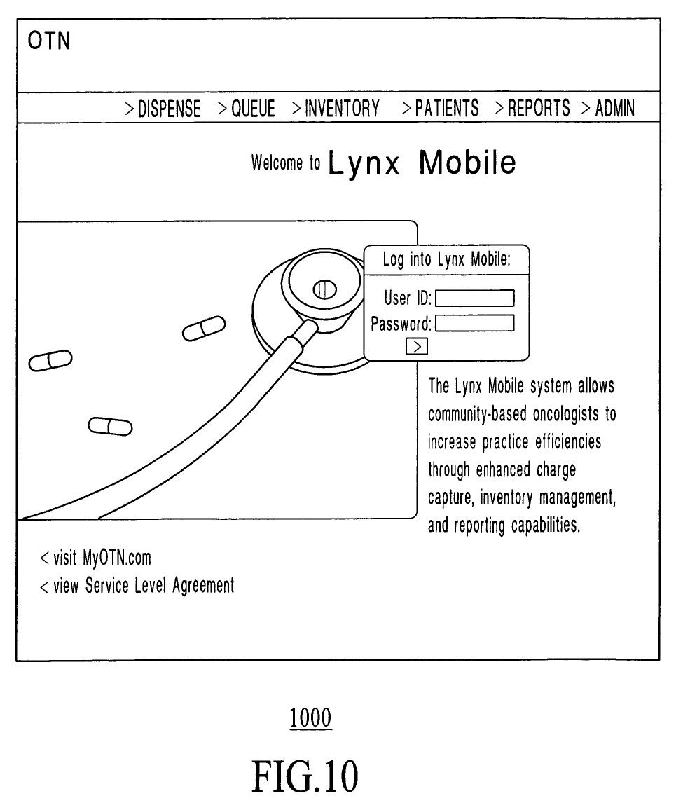

The Lynx Mobile Web site of an embodiment is secured at the HTTPS/Secure Sockets Layer (SSL) level and the application level via user identification and password. FIG. 10 is an example of a site login 1000 Web page or screen of the Lynx Mobile Web site, under an embodiment.

The Lynx Mobile system provides numerous classes of application accounts, including but not limited to system administrators, practice administrators, and other practice users. The system administrator account is assigned to internal users of the system, and provides a user with the authority to view or modify practice data, with the exception of identifying patient information, add new practices, and/or modify the master item list. The practice administrator account is assigned to one or two key practice users at the treatment facility, and provides a user with the authority to add new users to their practice and/or view or modify data for their practice; a user cannot view data for other practices via a practice administrator account. The other practice user account is assigned to billing personnel, and provides a user with the authority to view data for their practice; a user cannot view data for other practices via the other practice user account.

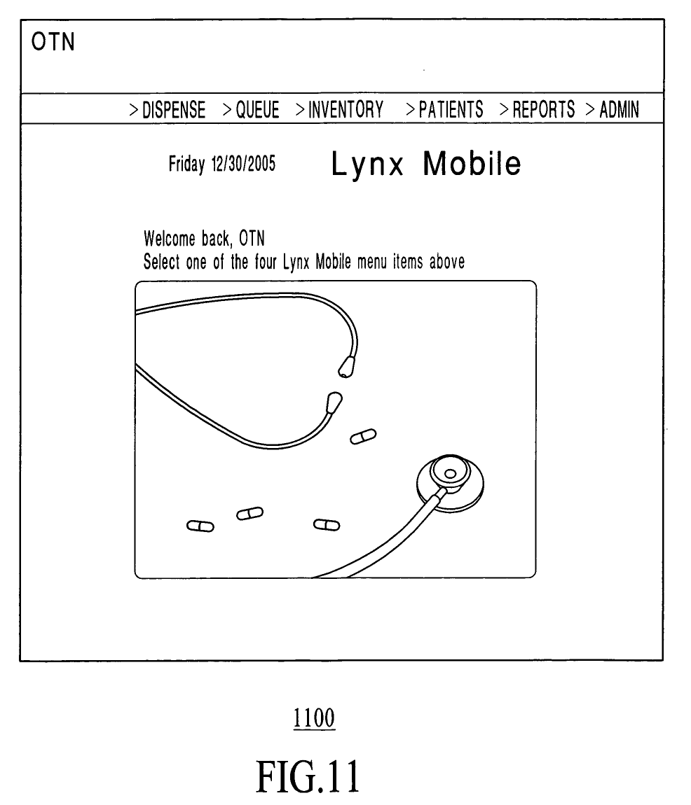

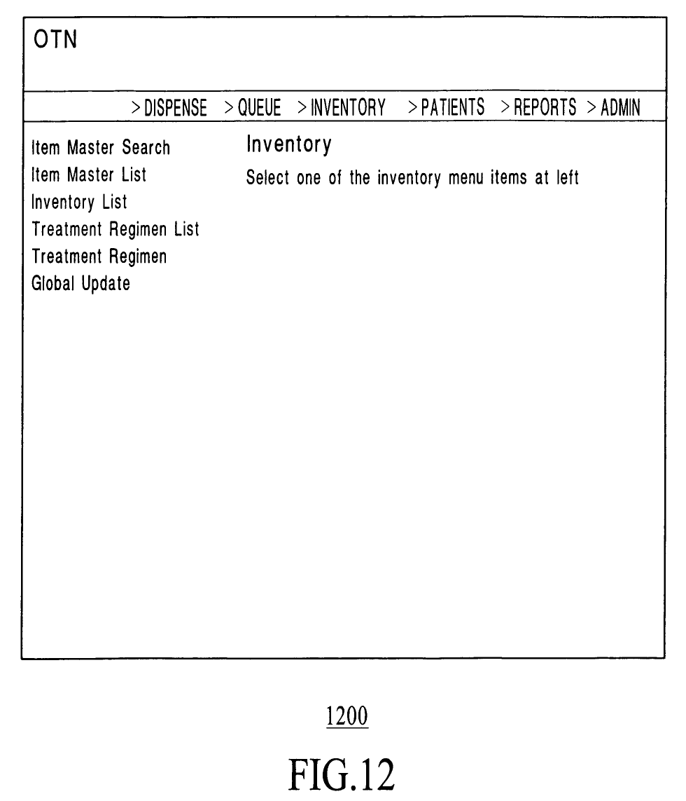

The Lynx Mobile Web site is organized into numerous areas that include but are not limited to Dispense, Queue, Inventory, Patients, Reports, and Administration to name a few. FIG. 11 is an example operations screen 1100 of a Lynx Mobile Web site, under an embodiment. Clicking or selecting any of the menus (“Dispense”, “Queue”, “Inventory”; “Patient”, “Reports”, “Admin”) in the upper portion of the frame (TopNav) changes the color of the box (e.g., changes the color of the box to dark blue) and displays a list of navigation options in the left-hand edge of the frame (SideNav). For example, FIG. 12 is an example operations screen 1200 of a Lynx Mobile Web Site following selection of the “Inventory” menu, under an embodiment.

Selecting a SideNav option (“Item Master List”, “Item Master Search”, “Inventory List”, Treatment Regimen List”, “Treatment Regimen Global Updates”) invokes that specific web site function. Not all functions may be available to all account classes. The above example includes use of a Practice Administrator-class account.

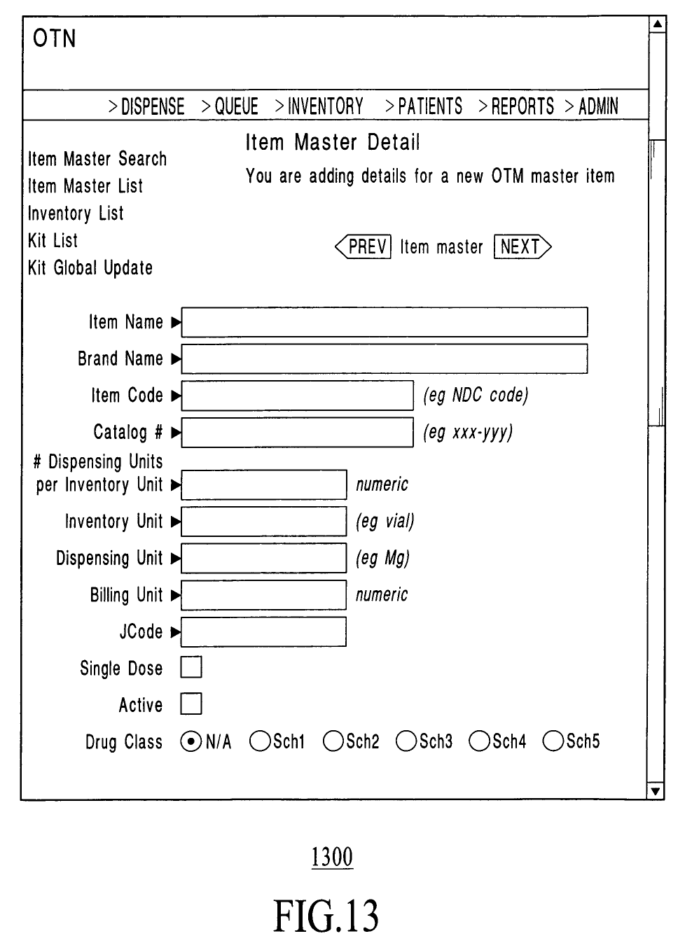

FIG. 13 is an example operations screen 1300 of a Lynx Mobile Web site showing the functions or selections available to a system administrator-class account, under an embodiment. The “Set Focus” link allows the System Administrator to place a specific practice “in focus”, that is, allow the System Administrator to become a Practice Administrator at that practice. This function is useful for trouble-shooting.

A user can access functions in the areas of inventory, patients, reports, and administration within each of the six organized areas of the Lynx Mobile Web site described above. Each of these functions is described below.

The inventory area of an embodiment includes but is not limited to an Item Master Search, Item Master List, Inventory List, Treatment Regimen List, and Treatment Regimen Global Update. The Item Master Search allows the user to use specific criteria to search for items in the item list. The Item Master List allows the practice to “activate” or “deactivate” specific items (products) from the master list of items. Activating an item places it into the practice inventory, and deactivating an item removes it from the practice inventory.

The Inventory List presents a summary list of all items in this practice's inventory. The user then has the option to click or select an item and view the detailed inventory information for that item. If this is a Practice Administrator-class account, the user can modify some inventory information for the item or can add a new inventory item which is not on the item master list. This menu also allows the user to Restock, Take inventory and\or Record Waste for non patient specific waste.

The Treatment Regimen List presents a summary list of all Treatment Regimen built by this practice. The user then has the option to click a Treatment Regimen name and view the inventory items that comprise that Treatment Regimen. If this is a Practice Administrator-class account, the user can modify the contents of the Treatment Regimen, delete the Treatment Regimen, or add a new Treatment Regimen.

The Treatment Regimen Global Update allows the user to Add, Replace and Remove items from existing Treatment Regimen.

The patient area of an embodiment includes but is not limited to a Patient Search, Patient List, and Patient Schedule. The Patient Search allows the user to use specific criteria to search the list of Lynx Mobile patients.

The Patient List presents a summary list of patients; the user then has the option to click a patient and view the detailed patient info. If this is a Practice Administrator-class account, the user can modify patient information, delete the patient, or add a new patient. If this is a System-Administrator-class account, the identifying patient information is obfuscated.

The Patient Schedule presents a calendar view of the current month. Links are present for the current day and all future days which allow the user to view the patient schedule for that day. If this is a Practice Administrator-class account, the user can add or delete patients from the schedule.

The reports area of an embodiment includes but is not limited to a Transaction Report, SuperBill Report, Refill Report, Treatment History, Item Usage History, and Item Audit to name a few. The Transaction Report presents start date and end date input fields which default to the current date. Upon entering a valid date range and clicking the “Run Report” button, a summary page is presented with links for every patient and date with treatment records (dispensed item records) within the given date range. Clicking or selecting the link presents a new browser window with a “printer-friendly” version of the detailed transactions for that specific patient and treatment date. The user can print the report using a “Send to Printer” button or via their browser's controls. The user also has the option to close the browser window using a “Close Window” button or via their browser's controls.

The SuperBill Report presents start date and end date input fields which default to the current date. Upon entering a valid date range and clicking the “Run Report” button, a summary page is presented with links for every patient and date with treatment records (dispensed item records) within the given date range. Links are also available to print all records found or to print all records for a given day. Clicking a link presents a new browser window with a “printer-friendly” version of a SuperBill-compatible report for the given patient(s) and treatment date(s). The user can print the report using a “Send to Printer” button or via their browser's controls. The user also has the option to close the browser window using a “Close Window” button or via their browser's controls.

The Refill Report shows the auto-refill items and the non-auto refill items that are to be reordered. The Treatment History is a report listing the treatment history for a specific patient(s) or all patients during a specific date range. The Treatment History is similar to a Patient flow sheet.

The Item Usage History is a report that displays the usage pattern of items over a period of time and does not include the usage of waste, restock, and/or changes to the inventory. The Item Audit is a report that displays the usage details of one or more inventory items over a span of time.

The administration area of an embodiment includes but is not limited to Practice Info, Doctor List, User List, ICD9 Codes, Ethnic Group List, Insurance List, Waste Reasons List, and Add a New Practice. The Practice Info displays practice-level data such as name, address and contact information. If this is a Practice Administrator-class account, the user can modify some practice information. If this is a System-Administrator-class account, the user can delete the practice.

The Doctor List presents a summary list of doctors at this practice. The user then has the option to click a doctor and view detailed doctor information. If this is a Practice Administrator-class account, the user can modify doctor information, delete the doctor, or add a new doctor.

The User List presents a summary list of users for this practice. The user then has the option to click a user and view the detailed user information. If this is an Other Practice User-class account and the user is viewing his/her own user record, he/she has the power to modify his/her password. If it is not his/her record, all fields are view-only. If this is a Practice Administrator-class account, the user can modify user information, delete users, or add new users. Under no circumstances is the password ever displayed, but the embodiment is not so limited.

The ICD9 Codes presents a list of ICD-9 diagnosis codes which can be assigned to a patient. If this is a Practice Administrator-class account, the user can modify the ICD-9 codes, delete ICD-9 codes, and/or add new ICD-9 codes.

The Ethnic Group List presents a list of ethnic groups which can be assigned to a patient. The Insurance List presents a list of insurance entities which can be assigned to a patient. If this is a Practice Administrator-class account, the user can modify the insurance entities, delete insurance entities, and/or add new insurance entities.

The Waste Reasons List presents a list of Waste reasons which can be used to attach to wasted items. The Add a New Practice allows System-Administrator-class users to add a new practice. A logout link is available in the upper-right-hand corner of every Web page of the Lynx Mobile Web site except the login page, but the embodiment is not so limited.

The Lynx Mobile system of an embodiment includes an order channel for automatically placing orders for inventory items like drugs and supplies with the appropriate vendors of these items. The order channel is referred to herein as the Lynx Mobile Order Channel. The Lynx Mobile Order Channel integrates with the Lynx Mobile system and functions to insert or place Lynx Mobile refill request messages into a Lynx Order Channel of the Lynx Mobile system. Raw refill request messages are provided by a Web Service on the Lynx Mobile Web site. The Lynx Mobile Order Channel additionally processes outbound messages (e.g., on-route, cancel).

The Lynx Mobile Order Channel of an embodiment generates one set of refill requests for a given treatment facility per day in order to keep the number of shipments to a treatment facility to as small a number as possible. Alternative embodiments may generate any number of sets of refill requests for a treatment facility.

The Lynx Mobile Order Channel of an embodiment includes numerous components that include an Order Channel Web site, a Message Translator (MsgTranslator), HouseKeeper, MidnightBackup, and Log Files to name a few. Each of these components is described in detail below.

The Order Channel Web site includes the use of one or more of Java (J2SE), Microsoft Internet Information Services (Web Server), Microsoft Active Server Pages, Microsoft SQL Server 2000 Relational Database Management System (RDBMS), Microsoft Windows Server 2003 Standard Edition (Operating System), but can include any type and/or combination of other technologies in the art.

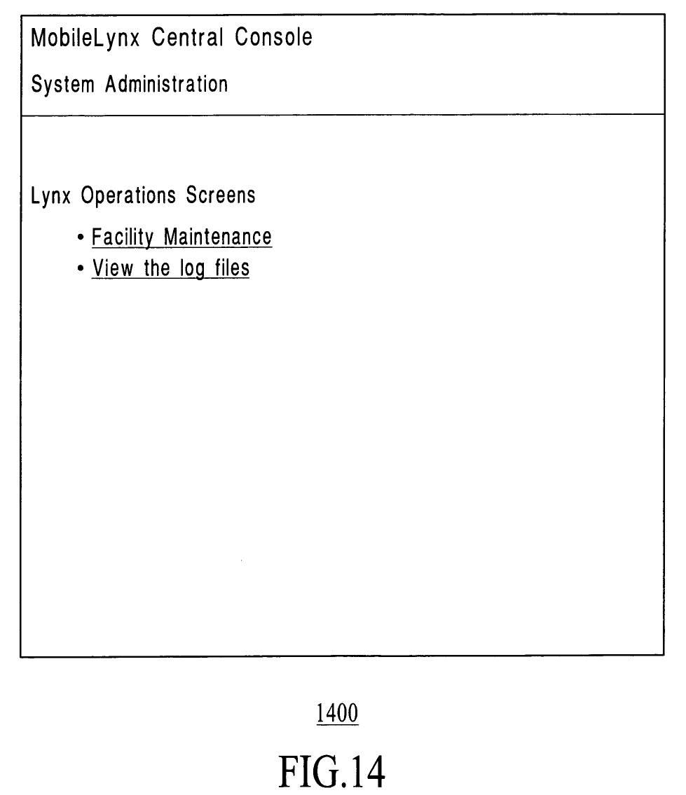

The Order Channel Web site of an embodiment includes a System Administration Web page or screen and a Facility Maintenance Web page or screen, but alternative embodiments may include any number/combination of screens. FIG. 14 is an example System Administration screen 1400 of the Order Channel Web site, under an embodiment. Clicking or selecting “View the log files” link leads to a directory of the various log files of the Lynx Mobile Order Channel.

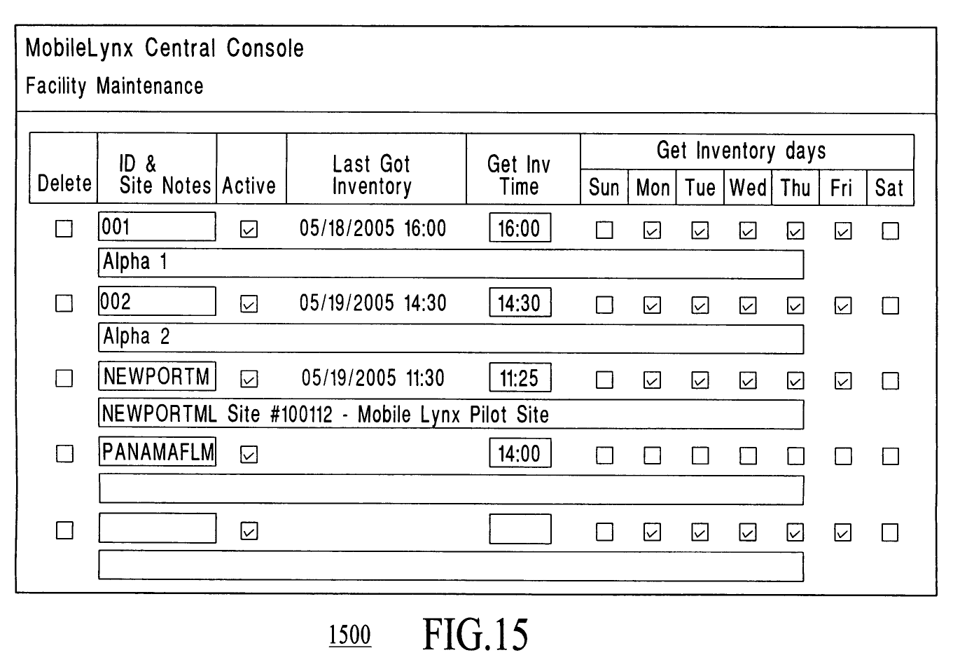

FIG. 15 is an example Facility Maintenance screen 1500 of the Order Channel Web site, under an embodiment. A user navigates to the Facility Maintenance Web page by selecting the “Facility Maintenance” link of the screen of the System Administration Web page. The Facility Maintenance screen allows the user to view, modify, or delete an existing facility or to add a new facility. Fields of the Facility Maintenance screen include but are not limited to Facility ID, Active, Last Got Inventory, Get Inv Time, Get Inventory Days, and Notes.

The Facility ID field ties the refill requests retrieved from the Lynx Mobile Web Site to a facility in the Lynx Order Channel. The Active field allows a user to appropriately mark a site as only sites marked “active” have their refill requests passed into the Lynx Order Channel.

The Last Got Inventory field is an informational field that indicates when the Lynx Mobile Order Channel last evaluated this facility to determine if refill requests should be injected into the Lynx Order Channel.

The Get Inv Time field indicates the time of day that the Lynx Mobile Order Channel should evaluate this facility to determine if refill requests should be injected into the Lynx Order Channel. The Get Inventory Days fields indicate day of the week that the Lynx Mobile Order Channel should evaluate this facility to determine if refill requests should be injected into the Lynx Order Channel. The Notes field is a free-form notes field.

The Message Translator of the Lynx Mobile Order Channel of an embodiment is run by the Windows Task Scheduler many times a day, but is not so limited. The term “MsgTranslator” as used herein describes both a specific program (MsgTranslatorjava), as well as a batch command file that runs many programs in series (MsgTranslator.bat). Unless otherwise indicated, the term MsgTranslator refers to the batch command file.

The Message Translator performs many operations as described below. The Message Translator builds a subdirectory under the “Logs” directory with a name in the YYYY.MM.DD format, representing the current date; other formats can be used in alternative embodiments. The Message Translator also starts a Java Virtual Machine (JVM) to invoke GetXml.class to obtain receipt (restock) messages from the Web Service on the Lynx Mobile Web site, starts a JVM to invoke LoadXml.class to load receipt messages into a MsgIncoming table, and starts a JVM to invoke GetXml.class to obtain refill request messages from the Web Service on the Lynx Mobile Web Site. Additionally, the Message Translator starts a JVM to invoke LoadXml.class to load refill request messages into the MsgIncoming table.

The Message Translator of an embodiment starts a JVM to invoke MsgTranslator.class to process outbound messages (from flat files generated by the Lynx Order Channel). For each on-route message, the Message Translator updates the corresponding record in the ItemBlock table, setting the purge date to five (5) days in the future, but the embodiment is not so limited.

The Message Translator of an embodiment starts a JVM to invoke MsgTranslator.class to process inbound messages (from the MsgIncoming table). For each inbound receipt message, operations delete the corresponding ItemBlock record, delete any accumulated refill request messages for this item for this facility, delete the receipt message from the MsgIncoming table, and write a receipt record in Lynx Order Channel format to a file. For every facility that is active, performs a “Get Inventory” on the current day of the week, has not had a “Get Inventory” performed on the current day, and whose “Get Inventory” time has been reached, the Message Translator of an embodiment starts a JVM to invoke MsgTranslator.class to process every refill request message for the current facility. Operations of the Message translator to process refill request messages include, but are not limited to, ignoring the refill request when there is an ItemBlock record for this item for this facility, building an ItemBlock record to correspond to this current item, deleting the refill request message from the MsgIncoming table, and writing a refill request record in Lynx Order Channel format to a file.

Any file created during inbound and outbound message processing is subsequently closed. The file has a name such that it will be consumed by the Lynx Order Channel.

The HouseKeeper of an embodiment performs various database and file system cleanup (housekeeping) duties. The HouseKeeper is run by Windows Task Scheduler once a day, late at night, and receives operating parameters via a configuration file, but is not so limited. The HouseKeeper performs numerous operations, as described below.

The HouseKeeper, for each directory in the list of “Purge Directories,” performs a recursive descent through the given directory tree, and deletes empty files that are older than the “Purge Days Empty Files” parameter. The HouseKeeper also performs a recursive descent through the given directory tree, searching for files matching the pattern “Receipt.*.xml” and, if any identified file(s) is older than the given “Purge Days” and file size matches a “Receipt File Purge Size”, deletes the file. The HouseKeeper also performs a recursive descent through the given directory tree, searching for files matching the pattern “Request.*.xml” and, if any identified file(s) is older than the given “Purge Days” and file size matches a “Request File Purge Size”, deletes the file.

For every record in the MsgIncoming table the HouseKeeper deletes the record when the accession date is older than the “Purge Days” parameter. For every record in the ItemBlock table the HouseKeeper deletes the record when the purge date is in the past.

The MidnightBackup of an embodiment includes a batch command file that copies the contents of database tables to the local file system. The MidnightBackup is run by the Windows Task Scheduler once a day, late at night, but is not so limited.

The Log Files of an embodiment can be browsed via the Order Channel Web Site. The general health of the Lynx Mobile Order Channel can be determined by examining these files. The Log Files are thus used for problem trouble-shooting.

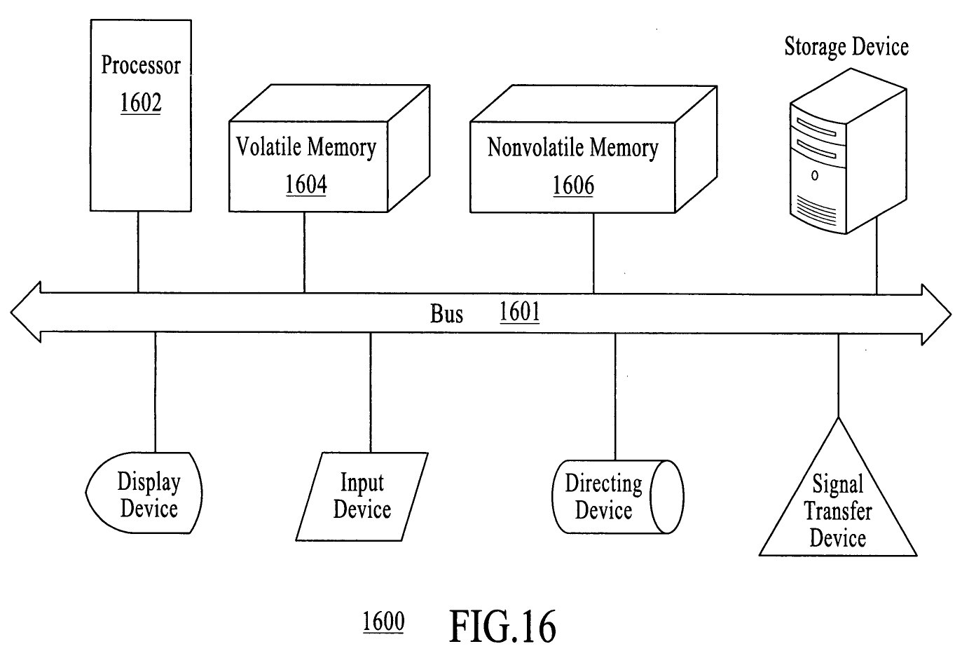

Although components of the Lynx Mobile system (e.g., portable devices 102-N, access points 110, and/or servers 130) may use a variety of different processing systems, FIG. 16 is a computer system 1600 for use in one or more of the Lynx Mobile system components, under an embodiment. The computer system 1600 generally includes a central processor unit (“CPU”) or central processor 1602 for processing information and instructions, an address/data bus 1601 coupled to the CPU 1602 for communicating information, volatile memory 1604 (random access memory (“RAM”) for example) coupled to the bus 1601 for storing information and instructions for the CPU 1602, and non-volatile memory 1606 (read-only memory (“ROM”) for example) coupled to the bus 1601 for storing static information and instructions for the CPU 1602. The computer system 1600 may also include one or more optional storage devices 1608 coupled to the bus 1601 for storing information and instructions. The storage devices or data storage devices 1608 can include one or more removable magnetic or optical storage media which are computer-readable memories. Some combination of the volatile memory 1604, non-volatile memory 1606, and/or storage device 1608 include or store data structures describing components or processes of the Lynx Mobile system described above, but the Lynx Mobile system is not limited to storage in these devices.

The computer system 1600 may also include at least one optional display device 1610 coupled to the bus 1601 for displaying information to the users of the computer system 1600. The computer system 1600 of an embodiment may also include one or more optional input devices 1612 coupled to the bus 1601 for communicating information and command selections to the CPU 1602. Additionally, the computer system 1600 may include an optional cursor control or directing device 1614 coupled to the bus 1601 for communicating user input information and command selections to the CPU 1602. The computer system 1600 may also include one or more optional signal transfer devices 1616 (transmitter, receiver, modem, antenna, etc. for example) coupled to the bus 1601 for interfacing with other computer systems.

The portable charge capture/inventory management systems and methods described above include a system comprising at least one server at a first location. The server of an embodiment includes inventory data and patient data. The inventory data of an embodiment includes information of drugs and supplies of a facility that is a patient treatment facility. The patient data of an embodiment includes identification, schedule and treatment data of each of a plurality of patients of the facility.

The system of an embodiment further includes at least one portable client at the facility that is coupled to the server. The portable client of an embodiment is configured to access and present the patient data for use during treatment of each patient. The portable client of an embodiment is configured to capture transaction data of the treatment at a point of care. The transaction data of an embodiment is automatically transferred to the server.

The transaction data of an embodiment includes data of one or more of drugs and supplies dispensed during the treatment, data of one or more of unused drugs and unused supplies resulting from the treatment, drugs and supplies wasted during the treatment, professional services of the treatment, administrative services of the treatment, current procedural terminology (CPT) codes of the treatment, evaluation and management (E&M) codes of the treatment, product codes of one or more of the drugs and supplies dispensed during the treatment.

The system of an embodiment includes a Web site hosted at the server and configured to allow manipulation of the inventory data and the patient data from one or more of the at least one portable client and at least one other computer of the facility.

The Web site of an embodiment is configured to allow manipulation of the patient data that includes one or more of generating and selecting a plurality of dispense profiles to control dispensing of one or more of drugs and supplies during the treatment.

The Web site of an embodiment is configured to provide access to the inventory data and the patient data of the facility.

The access of an embodiment is secure access and the inventory data and the patient data are accessed according to a plurality of categories comprising dispensing, queuing, inventory, patients, reports, and administration.

The server of an embodiment is configured to automatically update one or more of the inventory data and the patient data using the transaction data received from the at least one portable client.

The server of an embodiment is configured to automatically order one or more of drugs and supplies for the facility in response to one or more of the inventory data and the patient data.

The patient data of an embodiment includes billing data of the treatment received by a patient, wherein the server is configured to generate the billing data using the transaction data.

The server of an embodiment is further configured to automatically generate one or more of bills and billing reports for the facility in response to one or more of the inventory data and the patient data.

The system of an embodiment includes at least one network coupled to the server and the at least one portable client.

The system of an embodiment includes at least one inventory device at the facility coupled to one or more of the at least one server and the at least one portable client, the inventory device configured to one or more of contain and dispense drugs and supplies for use in the treatment.

The inventory device of an embodiment operates automatically in response to information received from one or more of the at least one server and the at least one portable client.

The first location of an embodiment is different from a location of the facility.

The portable client of an embodiment includes one or more of a portable computer (PC), personal digital assistant (PDA), communication device, and cellular telephone.

The portable charge capture/inventory management systems and methods described above include a method comprising receiving at a first location and storing inventory data and patient data. The inventory data of an embodiment includes information of drugs and supplies of a facility that is a patient treatment facility. The patient data of an embodiment includes identification, schedule and treatment data of each of a plurality of patients of the facility.

The method of an embodiment includes accessing and presenting the patient data at a point of care in the facility for use during treatment of each patient. The accessing and presenting of an embodiment is via at least one portable client and a coupling to the first location.

The method of an embodiment includes capturing transaction data of the treatment at the point of care during the treatment. The transaction data of an embodiment is automatically transferred to the first location.

The method of an embodiment further comprises automatically updating one or more of the inventory data and the patient data at the first location using the transaction data received from the at least one portable client.

The method of an embodiment further comprises automatically ordering one or more of the drugs and the supplies for the facility in response to one or more of the inventory data and the patient data at the first location.

The transaction data of an embodiment includes data of one or more of drugs and supplies dispensed during the treatment, data of one or more of unused drugs and unused supplies resulting from the treatment, drugs and supplies wasted during the treatment, professional services of the treatment, administrative services of the treatment, current procedural terminology (CPT) codes of the treatment, evaluation and management (E&M) codes of the treatment, product codes of one or more of the drugs and supplies dispensed during the treatment.

The method of an embodiment further comprises remotely manipulating the inventory data and the patient data stored at the first location.

The manipulating of an embodiment is via one or more of the at least one portable client and at least one other computer of the facility.

The manipulating of an embodiment is via a Web site of the first location.

The manipulating of an embodiment includes one or more of generating and selecting a plurality of dispense profiles to control dispensing of one or more of drugs and supplies during the treatment.

The accessing of an embodiment includes accessing the inventory data and the patient data of the facility.

The accessing of an embodiment includes accessing the inventory data and the patient data according to one or more of a plurality of categories comprising dispensing, queuing, inventory, patients, reports, and administration.

The method of an embodiment further comprises generating billing data from the transaction data, wherein the patient data includes billing data of the treatment received by a patient.

The method of an embodiment further comprises automatically generating at the first location one or more of bills and billing reports for the facility in response to one or more of the inventory data and the patient data.

The method of an embodiment further comprises automatically controlling at least one inventory device at the facility in response to information received from one or more of the at least one portable client and the first location, wherein the inventory device one or more of contains and dispenses drugs and supplies for use in the treatment.

The portable charge capture/inventory management systems and methods described above include a portable device comprising a processor configured to couple to at least one remote server. The portable device of an embodiment includes a user interface coupled to the processor and configured to access and present one or more of inventory data and patient data for use during treatment of a patient. The portable device of an embodiment is configured to capture transaction data of the treatment at a point of care in a facility. The portable device of an embodiment is configured to automatically transfer the transaction data to the remote server. The inventory data of an embodiment is at the remote server and includes information of drugs and supplies of the facility where the portable device is operating. The patient data of an embodiment includes identification, schedule and treatment data of each of a plurality of patients of the facility.

The portable charge capture/inventory management systems and methods described above include a device comprising at least one server at a location remote to a facility that is a patient treatment facility. The server of an embodiment is configured to transfer inventory data and patient data to and from a plurality of remote devices and store the inventory data and the patient data. The inventory data of an embodiment includes information of drugs and supplies of the facility. The patient data of an embodiment includes identification, schedule and treatment data of each of a plurality of patients of the facility. The server of an embodiment is configured to transfer to a portable client at the facility the patient data for use during treatment of a patient. The server of an embodiment is configured to receive transaction data of the treatment captured by the portable client at a point of care.

Aspects of the Lynx Mobile system and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the Lynx Mobile system and methods include: microcontrollers with memory (such as electronically erasable programmable read only memory (EEPROM)), embedded microprocessors, firmware, software, etc. Furthermore, aspects of the Lynx Mobile system and methods may be embodied in microprocessors having software-based circuit emulation, discrete logic (sequential and combinatorial), custom devices, fuzzy (neural) logic, quantum devices, and hybrids of any of the above device types. Of course any underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that the various processes and/or devices disclosed herein may be described using computer aided design tools and expressed (or represented), as data and/or instructions embodied in various computer-readable media, in terms of their behavioral, register transfer, logic component, transistor, layout geometries, and/or other characteristics. Formats of files and other objects in which such expressions may be implemented include, but are not limited to, formats supporting behavioral languages such as C, Verilog, and HLDL, formats supporting register level description languages like RTL, and formats supporting geometry description languages such as GDSII, GDSIII, GDSIV, CIF, MEBES and any other suitable formats and languages. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.).

When received within a computer system via one or more computer-readable media, such data and/or instruction-based expressions of the above described processes and/or devices may be processed by a processing entity (e.g., one or more processors) within the computer system in conjunction with execution of one or more other computer programs.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

The above description of illustrated embodiments of the Lynx Mobile system and methods are not intended to be exhaustive or to limit the processes and/or devices to the precise form disclosed. While specific embodiments of, and examples for, the Lynx Mobile system and methods are described herein for illustrative purposes, various equivalent modifications are possible within the scope of these processes and/or devices, as those skilled in the relevant art will recognize. The teachings of the Lynx Mobile system and methods provided herein can be applied to other processing systems and methods, not only for the systems and methods described above.

The elements and acts of the various embodiments described above can be combined to provide further embodiments. These and other changes can be made to the Lynx Mobile system and methods in light of the above detailed description.

In general, in the following claims, the terms used should not be construed to limit the Lynx Mobile system and methods to the specific embodiments disclosed in the specification and the claims, but should be construed to include all processing systems that operate under the claims. Accordingly, the Lynx Mobile system and methods are not limited by the disclosure, but instead the scope of the Lynx Mobile system and methods is to be determined entirely by the claims.

While certain aspects of the Lynx Mobile system and methods are presented below in certain claim forms, the inventors contemplate the various aspects of the Lynx Mobile system and methods in any number of claim forms. Accordingly, the inventors reserve the right to add additional claims after filing the application to pursue such additional claim forms for other aspects of the Lynx Mobile system and methods.

What is claimed is:

1. A system comprising:
at least one server at a first location, the server including inventory data and patient data, wherein the inventory data includes information of drugs and supplies of a facility that is a patient treatment facility, wherein the patient data includes identification, schedule and treatment data of each of a plurality of patients of the facility; and
at least one portable client at the facility that is coupled to the server, wherein the portable client is configured to access and present the patient data for use during treatment of each patient and configured to capture transaction data of the treatment at a point of care, wherein the transaction data is automatically transferred to the server.

2. The system of claim 1, wherein the transaction data includes data of one or more of drugs and supplies dispensed during the treatment, data of one or more of unused drugs and unused supplies resulting from the treatment, drugs and supplies wasted during the treatment, professional services of the treatment, administrative services of the treatment, current procedural terminology (CPT) codes of the treatment, evaluation and management (E&M) codes of the treatment, product codes of one or more of the drugs and supplies dispensed during the treatment.

3. The system of claim 1, further comprising a Web site hosted at the server and configured to allow manipulation of the inventory data and the patient data from one or more of the at least one portable client and at least one other computer of the facility.

4. The system of claim 3, wherein the Web site is configured to allow manipulation of the patient data that includes one or more of generating and selecting a plurality of dispense profiles to control dispensing of one or more of drugs and supplies during the treatment.

5. The system of claim 3, wherein the Web site is configured to provide access to the inventory data and the patient data of the facility.

6. The system of claim 5, wherein the access is secure access and the inventory data and the patient data are accessed according to a plurality of categories comprising dispensing, queuing, inventory, patients, reports, and administration.

7. The system of claim 1, wherein the server is configured to automatically update one or more of the inventory data and the patient data using the transaction data received from the at least one portable client.

8. The system of claim 1, wherein the server is configured to automatically order one or more of drugs and supplies for the facility in response to one or more of the inventory data and the patient data.

9. The system of claim 1, wherein the patient data includes billing data of the treatment received by a patient, wherein the server is configured to generate the billing data using the transaction data.

10. The system of claim 1, wherein the server is further configured to automatically generate one or more of bills and billing reports for the facility in response to one or more of the inventory data and the patient data.

11. The system of claim 1, further comprising at least one network coupled to the server and the at least one portable client.

12. The system of claim 1, further comprising at least one inventory device at the facility coupled to one or more of the at least one server and the at least one portable client, the inventory device configured to one or more of contain and dispense drugs and supplies for use in the treatment.

13. The system of claim 12, wherein the inventory device operates automatically in response to information received from one or more of the at least one server and the at least one portable client.

14. The system of claim 1, wherein the first location is different from a location of the facility.

15. The system of claim 1, wherein the at least one portable client includes one or more of a portable computer (PC), personal digital assistant (PDA), communication device, and cellular telephone.

16. A method comprising:
receiving at a first location and storing inventory data and patient data, wherein the inventory data includes information of drugs and supplies of a facility that is a patient treatment facility, wherein the patient data includes identification, schedule and treatment data of each of a plurality of patients of the facility;
accessing and presenting the patient data at a point of care in the facility for use during treatment of each patient, wherein the accessing and presenting is via at least one portable client and a coupling to the first location; and
capturing transaction data of the treatment at the point of care during the treatment, wherein the transaction data is automatically transferred to the first location.

17. The method of claim 16, further comprising automatically updating one or more of the inventory data and the patient data at the first location using the transaction data received from the at least one portable client.

18. The method of claim 16, further comprising automatically ordering one or more of the drugs and the supplies for the facility in response to one or more of the inventory data and the patient data at the first location.

19. The method of claim 18, wherein the transaction data includes data of one or more of drugs and supplies dispensed during the treatment, data of one or more of unused drugs and unused supplies resulting from the treatment, drugs and supplies wasted during the treatment, professional services of the treatment, administrative services of the treatment, current procedural terminology (CPT) codes of the treatment, evaluation and management (E&M) codes of the treatment, product codes of one or more of the drugs and supplies dispensed during the treatment.

20. The method of claim 18, further comprising remotely manipulating the inventory data and the patient data stored at the first location.

21. The method of claim 20, wherein the manipulating is via one or more of the at least one portable client and at least one other computer of the facility.

22. The method of claim 21, wherein the manipulating is via a Web site of the first location.

23. The method of claim 20, wherein the manipulating includes one or more of generating and selecting a plurality of dispense profiles to control dispensing of one or more of drugs and supplies during the treatment.

24. The method of claim 16, wherein the accessing includes accessing the inventory data and the patient data of the facility.

25. The method of claim 24, wherein the accessing includes accessing the inventory data and the patient data according to one or more of a plurality of categories comprising dispensing, queuing, inventory, patients, reports, and administration.

26. The method of claim 16, further comprising generating billing data from the transaction data, wherein the patient data includes billing data of the treatment received by a patient.

27. The method of claim 16, further comprising automatically generating at the first location one or more of bills and billing reports for the facility in response to one or more of the inventory data and the patient data.

28. The method of claim 16, further comprising automatically controlling at least one inventory device at the facility in response to information received from one or more of the at least one portable client and the first location, wherein the inventory device one or more of contains and dispenses drugs and supplies for use in the treatment.

29. A portable device comprising:
a processor configured to couple to at least one remote server; and
a user interface coupled to the processor and configured to access and present one or more of inventory data and patient data for use during treatment of a patient and configured to capture transaction data of the treatment at a point of care in a facility and automatically transfer the transaction data to the remote server, wherein the inventory data is at the remote server and includes information of drugs and supplies of the facility where the portable device is operating, wherein the patient data includes identification, schedule and treatment data of each of a plurality of patients of the facility.

30. A device comprising at least one server at a location remote to a facility that is a patient treatment facility, the server configured to transfer inventory data and patient data to and from a plurality of remote devices and store the inventory data and the patient data, wherein the inventory data includes information of drugs and supplies of the facility, wherein the patient data includes identification, schedule and treatment data of each of a plurality of patients of the facility, the server further configured to transfer to a portable client at the facility the patient data for use during treatment of a patient and to receive transaction data of the treatment captured by the portable client at a point of care.
